SEQUENCE LISTING (40 sequences)

SEQ ID NO: 1 - DNA, 2360 bp, Homo sapiens
gcgtcctcgcgggtctgaatggaagggtcgaggtcgtcgtcggcggcgagcagatcctgaagccagaactccaccccggcgcccgcgccatgcggcgggagaggtgcggcgccccccacccgcgtcgccgccatggaggtgctgcggcgctcctcggtcttcgccgccgagatcatggacgcctttgaccgctcgcccacagacaaggagctggtggcccaggccaaggcgctgggccgggagtacgtgcacgcgcggctgctgcgcgccggcctctcctggagcgcgcccgagcgtgccgcgccggtcccgggacgcctggctgaggtgtgcgcggtgctgctgcgcctgggcgatgagctggagatgatccggcccagcgtctaccgcaacgtggcgcgtcagctgcacatctccctgcagtctgagcctgtggtgaccgatgcgttcctggccgtggctggccacatcttctctgcaggcatcacgtggggcaaggtggtgtccctgtatgcggtggccgcggggctggccgtggactgtgtgaggcaggcccagcctgccatggtccacgccctcgtggactgcctgggggagttcgtgcgcaagaccctggcaacctggctgcggagacgcggcggatggactgatgtcctcaagtgtgtggtcagcacagaccctggcctccgctcccactggctggtggctgcactctgcagcttcggccgcttcctgaaggctgccttcttcgtgctgctgccagagagatgagctgcccacctggcagtggccgcagcctggccctctgggcccaacgcaggaggccctcagcacccgaacacatcttcctccgcagacccaggccctccggaaggggtgagtggggaggggctttcctgagcctggagctgggctttggggcagcctgcgaccctccccgcttgtgtcccttctcctgtgatctctgtgttttcccttttctttctggggccaggaagtcagggtcaactcccaggcctcagatgcaggggcccagaacacctgctctcacctgagccccaggtgaaggggcccgggaacacctgctctcacctgagccccaggtgaaggggcccgggaacacctgctctcacctgaaccccaggtgaaggggcccggaacacctgctctcacctgagccccaggtgaaggggcccggaacacctgctctcacctgagccccaggtgaaggggcccgggaacacctgctctcacctgagccccaggtgaaggggcccgggaacacctgctctcacctgaaccccaggtgaaggggcccggaacacctgctctcacctgagccccaggtgaaggggcccggaacacctgctctcacctgagccccaggtgaaggggcccgggaacacctgctctcacctgagcccctggtgaaggggcccggaacacttgctctcacctgagccccaggtgaaggggcccggaacacctgctctcacctgagccccaggtgaaggggcccggaacacctgctctcacctgagccccaggtgaaggggcccgggaacacctctcacctgaacccgggggtcccatcccaggaagaagggccatctcaggacatgagtcctcaggggccctgcacattcaatctgaaggtgaccctggcctggctgaagctggaagagctgtggggactcagcctgtaaacagagcgtaaggttcacatgctggttgcttaatccgtttctggaggaagagtatgacacccacttgtgatggggtccttgtgcggtggggaccggggccggcgggctccaggccagcacacctaacccatggatgtggaacctacggccgagaaggaatgttgcatgagtcggatcccagtccattgtcagtggagggtgagggtgaccccatctgctatttttgtgctcatcctcaaacaaccatttggggatgtgcctattagggctccgtaagaactcagatgcctgggaagcccagcccctcaggtgcccccacacacagccttcccttgacgcctacatttctaggcacatgtgaggcatctttcctggagccccgagccagccctgtccctccccagtgcagcatggcactcaggagatacaggctggacatggggcagtcgttctggggaggcctggcctagcagccacccacctgagccctcccggccaggcttcgtgctggggtgggccatgtgccaggacaggagggtcccggcggaaagccagccccggactcatcgtgacattgagatcccactggagggtaggggtggtaataaacttctccaaacgatcgttgtcattttaaaaaaaaaaaaaaaaaaa

SEQ ID NO: 2 - DNA, 642 bp, Homo sapiens
atggaggtgctgcggcgctcttcggtcttcgctgcggagatcatggacgcctttgatcgctggcccacagacaaggagctggtggcccaggctaaagcactaggccgggagtacgtgcacgcgcggcttttgcgcgccggcctctcctggagcgctccagagcgtgcctcgcctgcccctggaggacgcctggcagaggtgtgcaccgtgctgctgcgcttgggagatgagctggagcagatccgtcccagcgtatatcggaatgtggcccggcagctgcacatccctctgcagtctgagcctgtggtgactgatgccttcctcgctgtggccggccacatcttctcagcaggtatcacatggggcaaggtagtgtccctgtactcggcggctgcgggactagcggtggactgcgtccggcaagctcagccagccatggttcatgccctggttgactgcctgggggaatttgtacgcaagaccttggctacctggcttcggaggcgtggtggatggacggacgtcctcaagtgtgtggtcagcacaaaacctggcttccgctcccactggctcgtggccacactctgcagctttggccgcttcctgaaggctgcattcttcctcctgttgccagagagatga

SEQ ID NO: 3 - DNA, 642 bp, RAT
atggaggtgctgcggcgctcttctgtcttcgctgcggagatcatggacgcctttgatcgctcgcccacagacaaggagctggtggcccaggctaaagcactaggccgggagtacgtgcacgcgcggcttttgcgcgccggcctctcctggagcgctccagagcgtgcctcgcctgcccctggaggacgcctggcagaggtgtgcaccgtgctgctgcgcttgggagatgagctggagcagatccgtcccagcgtatatcggaatgtggcccggcagctgcacatccccctgcagtctgagcctgtggtgactgatgccttcctcgctgtggccggccacatcttctcagcaggtatcacatggggcaaggtagtgtccctgtactcggtggctgcgggactagcggtggactgcgtccggcaagctcagccagccatggttcatgccctggttgactgcctgggggaatttgtacgcaagaccctggccacctggcttcggaggcgtggtggatggacggacgtcctcaagtgtgtggtcagcacagaccctggcttccgctcccactggctcgtggccacactctgcagctttggccgcttcctgaaggctgcattcttcctcctgttgccagagagatga

SEQ ID NO: 4 - DNA, 1158 bp, chicken
ggggctgatggggatgggggcacagtggggcggtggaaccctacacacgttgacccagctctgccggaggagcaccgaaccatcccatcaggagccaccgagggggaccagagaccagcatgaagggtaaaatgaaaaaccgtctttgggagagcagctcctgggagctgtgatggagccccgtggatcgctgcttccctaaggctcagcccagcctggagctgggtggctttgctgcgaggatggaagtgctgcgccgctcgtccgtctttgctgcagaggtgatggaggtgttcgacaggtctcccactgacaaggagcttgtgtcccaagccaaggctctctgcagggactacatcaactccaggctgatccgtgcaggcgtcagctggagcaaacctgagcacaataccccggtgcctggaggcaagctggctgaggtgtctgccatcctgctgcgcctgggggatgagctggaatacattcgccccaacgtctaccgcaacatcgcccgccagctgaacatctcgctgcactcggagacggtggtgacggatgctttcctggccgtggctgcgcagatcttcactgcaggcataacatggggcaaggtggtgtctctctatgccgtggcagcggggctggcagtggactgcgtgcggcacgcacagccagccatggtccacaccatcgtggactgcctgggagagtttgtccgcaagaccttggtgacgtggctgaagaggcgaggaggctgggcagacatcaccaagtgcgtggtgagcaccgaccccagtctccgttcccactggctcgtggccgccgtctgcagctttgggcacttcctcaaggccatcttctttgtgctgctgcccgagagatgagccccaccgccctgtgccccatttcccccccgctccagaagcagtggccgtgggctctgaagaggaggaagagaagaactggtacagacacaacttgttctcctctcccttagctggtggcaagctgatggacttcattctcccttggatggccacagggagccctctcccaccgttcctgtcctaagaagccccatttcccatgggggggaccaagaagggagggaagggagcttattggattttggaaaacggggaatggggaaaaaggaaa

SEQ ID NO: 5 - DNA, 936 bp, Homo sapiens
ctcgtgccagcgtcctcgcgggtctgaatggaagggtcgaggtcgtcgtcggcggcgagcagatcctgaagccagaactccaccccggcgcccgcgccatgcggcgggagaggtgcggcgccccccacccgcgtcgccgccatggaggtgctgcggcgctcctcggtcttcgccgccgagatcatggacgcctttgaccgctcgcccacagacaaggagctggtggcccaggccaaggcgctgggccgggagtacgtgcacgcgcggctgctgcgcgccggcctctcctggagcgcgcccgagcgtgccgcgccggtcccgggacgcctggctgaggtgtgcgcggtgctgctgcgcctgggcgatgagctggagatgatccggcccagcgtctaccgcaacgtggcgcgtcagctgcacatctccctgcagtctgagcctgtggtgaccgatgcgttcctggccgtggctggccacatcttctctgcaggcatcacgtggggcaaggtggtgtccctgtatgcggtggccgcggggctggccgtggactgtgtgaggcaggcccagcctgccatggtccacgccctcgtggactgcctgggggagttcgtgcgcaagaccctggcaacctggctgcggagacgcggcggatggactgatgtcctcaagtgtgtggtcagcacagaccctggcctccgctcccactggctggtggctgcactctgcagcttcggccgcttcctgaaggctgccttcttcgtgctgctgccagagagatgagctgcccacctggcagtggccgcagcctggccctctgggcccaacgcaggaggccctcagcacccgaacacatcttcctcctccccacccgagcctggagcactctaacctcggagaccccctaagccccgttcctccgcagacccaggccctccg

SEQ ID NO: 6 - DNA, 720 bp, chicken
gctgcttccctaaggctcagcccagcctggagctgggtggctttgctgcgaggatggaagtgctgcgccgctcgtccgtctttgctgcagaggtgatggaggtgttcracaggtctcccactgacaaggagcttgtgtcccaagccaaggctctctgcagggactacatcaactccaggctgatccgtrcaggcgtcagctggagcaaacctgagcacaataccccggtgcctggaggcaagctggctgaggtgtctgccatcctgctgcgcctgggggatgagctggaatacattcgccccaacgtctaccgcaacatcgcccgccagctgaacatctcgctgcactcggagacggtggtgacggatgctttcctggccgtggctgcgcagatcttcactgcaggcataacatggggcaaggtggtgtctctctatgccgtggcagcggggctggcagtggactgcgtgcggcacgcacagccagccatggtccacaccatcgtggactgcctgggagagtttgtccgcaagaccttggtgacgtggctgaagaggcgaggaggctgggcagacatcaccaagtgcgtggtgagcaccgaccccagtctccgttcccactggctcgtggccgccgtctgcagctttgggcacttcctcaaggccatcttctttgtgctgctgcccgagagatgagccccaccgccctgtgccccatttc

SEQ ID NO: 7 - DNA, 1618 bp, Drosophila
gagagggtggtaggccgattccctctccccactgcccgttgaaattcagaatactaagctctcggttaaacgcggcgaaaaagaaagcaagctctgagcggctgaaaaaaaaatgaagtgaaataaaactgggatcgcggcaccagcaacaagttttagtggctcttctttgtgcgtttcgttcgtgtttgctgccctgcgctttgctcgccacattcgtcgccgacttttattctgttttgcccattttatcagaatcggagcacctccaaaaaagcccaagacgagctgagcctcagctgcgtcgaggtgagctgatccactccgctcccctttcgtgcgctgcccaccgctccccaccgctcacacccgatcccatccaatccaatccgatccgctccgctccgagtgcatagtgcatgcaaagtcgcggggctgggctttcggaattacacaacccacatggggcagcgccaacaaaggcctcagcagcaacaatagcgggcagccaagcattgccctgccctcgacttcgaccatggctcccaccaccagtccgccacccaagctggccaagttcaagtcctcgtcgctggaccacgagatctacacggccaatcgccgcggcaccattgccacggcctccagcgactggaaggcgctccgcggaggcgtcggtggaggagcaggaggacccggtagcgtacccaatccctctaacggacgctcccttcacgccggcggacccatgacacgggccgcctccacatcctcgctggctagcagtacgcgcacgatgactaactaccaggagtacaaaatggatatcatcaaccaggggaaatgtctgtgtggtcagtacatcagagcgcggctgcgacgggcaggagtcctcaaccggaaggtgacacagcgtttgcgcaacatcctggaccccggctcctcgcacgtggtctatgaagttttcccggcactgaacagcatgggcgaggaactggagcggatgcacccgcgggtgtacacaaacatatcgcgacagctgtcgagggccccgtttggcgagctggaggacagcgacatggcgcccatgttgctcaacctagttgccaaggatctttttcgctccagcatcacctggggcaagataatctcgatatttgccgtatgcggcggctttgccatagactgcgtgcgccagggacatttcgactacctacagtgcctgattgacggtctggctgagatcataggacgacctggtctactggctgatcgacaacggcggatggttgggcctgtcgcggcacatccgaccccgggtcggcgaatttacgttcttgggatggttgacgctgttcgtgactatctctgcaggcgcatatatggtctcaaacgtgtgtcggcgcattggaggtcaactgtattcgctgctgttctagattcgcttgggatcgcgtcgttaagaaatacaatcgtaccatttagtcaatgagagcttcaaatcattcctgcttccatgggcaccagtcgtttagtagtatgtaacggaccctgttttacgtataatattgttattccctttctcctctttttgtacatacaaggctattctaggcgc

SEQ ID NO: 8 - DNA, 642 bp, Homo sapiens; identical to SEQ ID NO: 2

SEQ ID NO: 9 - DNA, 513 bp, RAT
atggaggtgctgcggcgctcttctgtcttcgctgcggagatcatggacgcctttgatcgctcgcccacagacaaggagctggtggcccaggctaaagcactaggccgggagtacgtgcacgcgcggcttttgcgcgccggcctctcctggagcgctccagagcgtgcctcgcctgcccctggaggacgcctggcagaggtgtgcaccgtgctgctgcgcttgggaatcacatggggcaaggtagtgtccctgtactcggtggctgcgggactagcggtggactgcgtccggcaagctcagccagccatggttcatgccctggttgactgcctgggggaatttgtacgcaagaccctggccacctggcttcggaggcgtggtggatggacggacgtcctcaagtgtgtggtcagcacagaccctggcttccgctcccactggctcgtggccacactctgcagctttggccgcttcctgaaggctgcattcttcctcctgttgccagagagatga

SEQ ID NO: 10 - DNA, 642 bp, RAT; identical to SEQ ID NO: 3

SEQ ID NO: 11 - protein, 212 aa, Homo sapiens
MEVLRRSSVFAAEIMDAFDRSPTDKELVAQAKALGREYVHARLLRAGLSWSAPERAAPVPGRLAEVCAVLLRLGDELEMIRPSVYRNVARQLHISLQSEPVVTDAFLAVAGHIFSAGITWGKVVSLYAVAAGLAVDCVRQAQPAMVHALVDCLGEFVRKTLATWLRRRGGWTDVLKCVVSTDPGLRSHWLVAALCSFGRFLKAAFFVLLPER

SEQ ID NO: 12 - protein, 213 aa, Homo sapiens
MEVLRRSSVFAAEIMDAFDRWPTDKELVAQAKALGREYVHARLLRAGLSWSAPERASPAPGGRLAEVCTVLLRLGDELEQIRPSVYRNVARQLHIPLQSEPVVTDAFLAVAGHIFSAGITWGKVVSLYSAAAGLAVDCVRQAQPAMVHALVDCLGEFVRKTLATWLRRRGGWTDVLKCVVSTKPGFRSHWLVATLCSFGRFLKAAFFLLLPER

SEQ ID NO: 13 - protein, 213 aa, RAT
MEVLRRSSVFAAEIMDAFDRSPTDKELVAQAKALGREYVHARLLRAGLSWSAPERASPAPGGRLAEVCTVLLRLGDELEQIRPSVYRNVARQLHIPLQSEPVVTDAFLAVAGHIFSAGITWGKVVSLYSVAAGLAVDCVRQAQPAMVHALVDCLGEFVRKTLATWLRRRGGWTDVLKCVVSTDPGFRSHWLVATLCSFGRFLKAAFFLLLPER

SEQ ID NO: 14 - protein, 213 aa, chicken
MEVLRRSSVFAAEVMEVFDRSPTDKELVSQAKALCRDYINSRLIRAGVSWSKPEHNTPVPGGKLAEVSAILLRLGDELEYIRPNVYRNIARQLNISLHSETVVTDAFLAVAAQIFTAGITWGKVVSLYAVAAGLAVDCVRHAQPAMVHTIVDCLGEFVRKTLVTWLKRRGGWADITKCVVSTDPSLRSHWLVAAVCSFGHFLKAIFFVLLPER

SEQ ID NO: 15 - protein, 212 aa, Homo sapiens; identical to SEQ ID NO: 11

SEQ ID NO: 16 - protein, 213 aa, chicken
  MOD_RES (46): xaa = anything
MEVLRRSSVFAAEVMEVFBRSPTDKELVSQAKALCRDYINSRLIRXGVSWSKPEHNTPVPGGKLAEVSAILLRLGDELEYIRPNVYRNIARQLNISLHSETVVTDAFLAVAAQIFTAGITWGKVVSLYAVAAGLAVDCVRHAQPAMVHTIVDCLGEFVRKTLVTWLKRRGGWADITKCVVSTDPSLRSHWLVAAVCSFGHFLKAIFFVLLPER

SEQ ID NO: 17 - protein, 317 aa, Drosophila
MAPTTSPPPKLAKFKSSSLDHEIYTANRRGTIATASSDWKALRGGVGGGAGGPGSVPNPSNGRSLHAGGPMTRAASTSSLASSTRTMTNYQEYKMDIINQGKCLCGQYIRARLRRAGVLNRKVTQRLRNILDPGSSHVVYEVFPALNSMGEELERMHPRVYTNISRQLSRAPFGELEDSDMAPMLLNLVAKDLFRSSITWGKIISIFAVCGGFAIDCVRQGHFDYLQCLIDGLAEIIGRPGLLADRQRRMVGPVAAHPTPGRRIYVLGMVDAVRDYLCRRIYGLKRVSAHWRSTVFAAVLDSLGIASLRNTIVPFSQ

SEQ ID NO: 18 - protein, 213 aa, Homo sapiens; identical to SEQ ID NO: 12

SEQ ID NO: 19 - protein, 170 aa, RAT
MEVLRRSSVFAAEIMDAFDRSPTDKELVAQAKALGREYVHARLLRAGLSWSAPERASPAPGGRLAEVCTVLLRLGITWGKVVSLYSVAAGLAVDCVRQAQPAMVHALVDCLGEFVRKTLATWLRRRGGWTDVLKCVVSTDPGFRSHWLVATLCSFGRFLKAAFFLLLPER

SEQ ID NO: 20 - protein, 213 aa, RAT; identical to SEQ ID NO: 13

SEQ ID NO: 21 - DNA, 1430 bp, Mus musculus
ggggcggactgtgcttcagctcgggtgtggacggggcgggcgctggggcggggcgccgctcgcgggtttgaatggaagggtctagaccgccggagacgcagcgagcgggtcctgaaaccagaactccaccgccgccccgcgcgccatgaggcgggagaggtgtggcgcctctcgcccgcgcttcggccatggaggtgctgcggcgctcttctgtctttgcagcggagatcatggacgcctttgatcgctcgcccacagacaaggagctggtggcccaggctaaggcactaggccgggagtacgtgcacgcgcggcttttgcgcgccggcctctcctggagcgctccagagcgtgcctcgcctgcccctggaggacgcttggcagaggtgtgcacagtgctgctgcgcttgggagatgagctggagcagatccgtcccagcgtataccggaacgtggcccggcagctgcacatccccctgcagtcggagcctgtggtgacggatgccttcctggcagtggcaggccacatcttctcagcaggtatcacatggggcaaggtagtgtccctgtattccgtggccgcggggctagcggtggactgtgtccggcaggctcagcctgccatggttcatgccctggttgactgcctgggggagtttgtacgcaagaccttggctacctggcttcggaggcgtggtggatggacggatgtcctcaagtgtgtggtcagcacagatcctggcttccgctcccactggctcgtggccacgctctgcagctttggccgcttcctgaaggcagcattcttcctgttgttgccagagagatgagctggccaccagggcaggggccactcctagggtccctgggcccaatccaaggggcctccagtacctacaaagcacctccctcactcaaattgggagcatttagcccctggggccctgtcccaaacccattcctttgtggaccctggcctctgagagaggagtgtggagaaagccagagtctggagctggcctctgtgactgctctagctcttctcctggaactcctgccagaaagtcagggtcggtgtccagccctagagaaagggacctgtgaatactttcactcgatttcccaggccccacccaaggaaggggccttccccagacatgagctggcctcagtctttgtggaaagcaggtcctgtacatttgacctgaaggggactctggctaatgcaggagaagccagggatgcagagtaagagagcttcttgcttagggtatttttagatgaagaaggtatccctaagccacgatgggtccttcatggcaggaaccagagaggtagggttttagggtagttcacctgaccaatgaacaagagatcctgtggatgagggggtttgtataagttaaaatccaataaagctttacctagtg

SEQ ID NO: 22 - DNA, 1430 bp, Mus musculus; identical to SEQ ID NO: 21

SEQ ID NO: 23 - DNA, 2353 bp, Homo sapiens
ggcacgagggaatggaagggtcgaggtcgtcgtcggcggcgagcagatcctgaagccagaactccaccccggcgcccgcgccatgcggcgggagaggtgcggcgccccccacccgcgtcgccgccatggaggtgctgcggcgctcctcggtcttcgccgccgagatcatggacgcctttgaccgctcgcccacagacaaggagctggtggcccaggccaaggcgctgggccgggagtacgtgcacgcgcggctgctgcgcgccggcctctcctggagcgcgcccgagcgtgccgcgccggtcccgggacgcctggctgaggtgtgcgcggtgctgctgcgcctgggcgatgagctggagatgatccggcccagcgtctaccgcaacgtggcgcgtcagctgcacatctccctgcagtctgagcctgtggtgaccgatgcgttcctggccgtggctggccacatcttctctgcaggcatcacgtggggcaaggtggtgtccctgtatgcggtggccgcggggctggccgtggactgtgtgaggcaggcccagcctgccatggtccacgccctcgtggactgcctgggggagttcgtgcgcaagaccctggcaacctggctgcggagacgcggcggatggactgatgtcctcaagtgtgtggtcagcacagaccctggcctccgctcccactggctggtggctgcactctgcagcttcggccgcttcctgaaggctgccttcttcgtgctgctgccagagagatgagctgcccacctggcagtggccgcagcctggccctctgggcccaacgcaggaggccctcagcacccgaacacatcttcctccgcagacccaggccctccggaaggggtgagtggggaggggctttcctgagcctggagctgggctttggggcagcctgcgaccctccccgcttgtgtcccttctcctgtgatctctgtgttttcccttttctttctggggccaggaagtcagggtcaactcccaggcctcagatgcaggggcccagaacacctgctctcacctgagccccaggtgaaggggcccgggaacacctgctctcacctgagccccaggtgaaggggcccgggaacacctgctctcacctgaaccccaggtgaaggggcccggaacacctgctctcacctgagccccaggtgaaggggcccggaacacctgctctcacctgagccccaggtgaaggggcccgggaacacctgctctcacctgagccccaggtgaaggggcccgggaacacctgctctcacctgaaccccaggtgaaggggcccggaacacctgctctcacctgagccccaggtgaaggggcccggaacacctgctctcacctgagccccaggtgaaggggcccgggaacacctgctctcacctgagcccctggtgaaggggcccggaacacttgctctcacctgagccccaggtgaaggggcccggaacacctgctctcacctgagccccaggtgaaggggcccggaacacctgctctcacctgagccccaggtgaaggggcccgggaacacctctcacctgaacccgggggtcccatcccaggaagaagggccatctcaggacatgagtcctcaggggccctgcacattcaatctgaaggtgaccctggcctggctgaagctggaagagctgtggggactcagcctgtaaacagagcgtaaggttcacatgctggttgcttaatccgtttctggaggaagagtatgacacccacttgtgatggggtccttgtgcggtggggaccggggccggcgggctccaggccagcacacctaacccatggatgtggaacctacggccgagaaggaatgttgcatgagtcggatcccagtccattgtcagtggagggtgagggtgaccccatctgctatttttgtgctcatcctcaaacaaccatttggggatgtgcctattagggctccgtaagaactcagatgcctgggaagcccagcccctcaggtgcccccacacacagccttcccttgacgcctacatttctaggcacatgtgaggcatctttcctggagccccgagccagccctgtccctccccagtgcagcatggcactcaggagatacaggctggacatggggcagtcgttctggggaggcctggcctagcagccacccacctgagccctcccggccaggcttcgtgctggggtgggccatgtgccaggacaggagggtcccggcggaaagccagccccggactcatcgtgacattgagatcccactggagggtaggggtggtaataaacttctccaaacgatcgttgtcattttaaaaaaaaaaaaaaaaaaa

SEQ ID NO: 24 - protein, 213 aa, Mus musculus; identical to SEQ ID NO: 13

SEQ ID NO: 25 - protein, 213 aa, Mus musculus; identical to SEQ ID NO: 13

SEQ ID NO: 26 - protein, 212 aa, Homo sapiens; identical to SEQ ID NO: 11

SEQ ID NO: 27 - protein, 213 aa, RAT; identical to SEQ ID NO: 13

SEQ ID NO: 28 - protein, 213 aa, Homo sapiens; identical to SEQ ID NO: 12

SEQ ID NO: 29 - protein, 170 aa, RAT; identical to SEQ ID NO: 19

SEQ ID NO: 30 - protein, 170 aa, Homo sapiens
MEVLRRSSVFAAEIMDAFDRWPTDKELVAQAKALGREYVHARLLRAGLSWSAPERASPAPGGRLAEVCTVLLRLGITWGKVVSLYSAAAGLAVDCVRQAQPAMVHALVDCLGEFVRKTLATWLRRRGGWTDVLKCVVSTKPGFRSHWLVATLCSFGRFLKAAFFLLLPER

SEQ ID NO: 31 - protein, 13 aa, Artificial Sequence
  Description of Artificial Sequence  Synthetic Peptide
LRRAGDEFERYRR

SEQ ID NO: 32 - protein, 12 aa, RAT
LLRLGDELEQIR

SEQ ID NO: 33 - protein, 12 aa, Homo sapiens
LLRLGDELEQIR

SEQ ID NO: 34 - protein, 212 aa, Artificial Sequence
  Description of Artificial Sequence  Synthetic Peptide
MEVLRRSSVFAAEIMDAFDRSPTDKELVAQAKALGREYVHARLLRAGLSWSAPERAAPVPGRLAEVCAVLAAAGDELEMIRPSVYRNVARQLHISLQSEPVVTDAFLAVAGHIFSAGITWGKVVSLYAVAAGLAVDCVRQAQPAMVHALVDCLGEFVRKTLATWLRRRGGWTDVLKCVVSTDPGLRSHWLVAALCSFGRFLKAAFFVLLPER

SEQ ID NO: 35 - DNA, 18 bp, Artificial Sequence
  Description of Artificial Sequence  Synthetic Primer
ctggcggcggcgggcgat

SEQ ID NO: 36 - protein, 12 aa, Homo sapiens
TVLLRLGDELEM

SEQ ID NO: 37 - protein, 9 aa, Homo sapiens
LLRLGDELE

SEQ ID NO: 38 - DNA, 27 bp, Homo sapiens
gcctttgaccgctcgcccacagacaag

SEQ ID NO: 39 - DNA, 27 bp, Artificial Sequence
  Description of Artificial Sequence  Synthetic Primer
gcctttgaccgcgcgcccacagacaag

SEQ ID NO: 40 - DNA, 27 bp, Artificial Sequence
  Description of Artificial Sequence  Synthetic Primer
gaccgctcgcccgcggacaaggagctg

(19) United States
(12) Patent Application Publication
(10) Pub. No.: US 2003/0139344 A1
(43) Pub. Date: Jul. 24, 2003

(54) Antitumor activity of Bok

(76) Inventors: Mien-Chie Hung, Houston, TX (US); Yiyu Zou, Houston, TX (US); Ka Yin Kwong, Rockville, MD (US); Geoffrey Bartholomeusz, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/299,514

(22) Filed: Nov. 19, 2002

Related U.S. Application Data
(60) Provisional application No. 60/331,699, filed on Nov. 19, 2001.

(51) Int. Cl. A61K 48/00; A61K 9/127; C12N 15/86; C12N 15/88; A61K 38/17

(52) U.S. Cl. 514/12; 514/44; 435/455; 435/456; 435/458; 424/93.2; 424/450

(57) ABSTRACT

The present invention is directed to the pro-apoptotic bok gene product that provides antitumor activity, particularly through induction of apoptosis. In some embodiments of the present invention, Bok is utilized for ovarian cancer, among others, and the composition can be delivered by either viral or non-viral delivery methods.

This application claims priority to U.S. Provisional Patent Application Serial No. 60/331,699, filed Nov. 19, 2001, which is incorporated by reference herein in its entirety.

### FIELD OF THE INVENTION

The present invention is directed to the fields of cancer, cell biology, and molecular biology. More specifically, the present invention relates to methods regarding the antitumor activity of Bcl-2-related ovarian killer (Bok).

### BACKGROUND OF THE INVENTION

Apoptosis, or programmed cell death, is important during many cellular events, including metamorphosis, hormone-induced tissue atrophy, embryonic development, and tissue renewal. Furthermore, apoptosis plays a pivotal role in self-defense against cellular transformation and tumor development by eliminating superfluous and/or damaged cells without eliciting inflammatory response, as opposed to necrosis.

Apoptosis is considered to entail two series of events, namely decision and execution. In the initial decision steps, significant players include the Bcl-2 family of proteins consisting of different anti- and pro-apoptotic members. The execution phase of apoptosis is mediated by the activation of caspases, which are cysteine proteases that act through proteolytic cleavage of substrates important for cellular homeostasis.

The life-or-death fate of a cell is determined by the highly regulated specific interactions among the different members of the Bcl-2 family that includes antiapoptotic proteins, proapoptotic proteins, and proapoptotic ligands. Bok is a proapoptotic protein of the Bcl-2 family. Bok interacts strongly with some (Mcl-1, BHRF1, and Bfl-1) but not other (Bcl-2, Bcl-xL, and Bcl-w) anti-apoptotic members, which is in direct contrast to other pro-apoptotic members (Bax, Bak, and Bik) which interact with all of the Bcl-2 family anti-apoptotic proteins. Thus, the key difference between Bok protein and other proapoptotic members of the Bcl-2 family, such as Bax and Bak, is that Bok-induced apoptosis is not inhibited by Bcl-2, an antiapoptotic protein. Since Bcl-2 is amplified or over-expressed in many human cancers, the apoptotic activity of other pro-apoptosis genes are counterreacted by Bcl-2, and thus can not effectively exert their antitumor activity. In contrast, bok can induce apoptosis in Bcl-2 over-expressed cancer cells. However, the possibility that the bok gene acts as a tumor suppressor gene has never been directly tested.

Hsu et al. (1997) identified Bok as a pro-apoptotic Bcl-2 family member having restricted expression in the ovary, particularly granulosa cells, testis and uterus, and that it heterodimerizes with selective anti-apoptotic Bcl-2 family members:Mcl-1, BHRF1, Bfl-1, but not Bcl-2, Bcl-xL, or Bcl-w. However, Hsu and Hsueh (1998) describe a splicing variant of Bok having a truncated BH3 domain, the heterodimerization motif, which still induces apoptosis but does not dimerize with the antiapoptotic Bcl-2 proteins Mcl-1, BHRF-1, and Bfl-1 in vitro.

U.S. Pat. Nos. 6,043,055; 6,222,017; and 6,437,097, in addition to Hsu et al. (1997) and Hsu and Hsueh (1998), are directed to Bok compositions. Furthermore, U.S. Pat. No. 6,376,247 regards using Bok compositions in methods to induce apoptosis. Although these references address Bok compositions for apoptosis, such as regarding follicular atresia (see related review by Hsu and Hsueh (2000)), they do not demonstrate to a skilled artisan in an enabled manner that the Bok compositions are useful for cancer treatment nor methods of using such Bok compositions for cancer treatment. Particularly, these patents contain no data indicating that Bok compositions can be used to treat cancer, in contrast to the present specification. Furthermore, there is no teaching of a Bok nuclear localization activity coupled to an apoptosis function, and they lack teaching of a specific Bok mutant comprising enhanced activity over wild type.

### SUMMARY OF THE INVENTION

Bok is a proapoptotic member of the Bcl-2 family of proteins. Although Bok has been previously shown to be associated with apoptotic activity, such as with follicular atresia and in other reproductive tissues, it has never been reported in an enabling manner to have anti-tumor activity. In the present invention, the inventors demonstrate the novel finding that both rat and human wild type versions and at least one mutant human version of a bok polynucleotide exerted strong antitumor activity in both in vivo and in vitro experimental systems. The Examples presented herein indicate that the transfected bok polynucleotide significantly induced apoptosis in various human cancer cells, especially in human ovarian, pancreatic, breast, and/or prostate cancer cells. More importantly, intraperitoneal injections of a bok polynucleotide in a nonviral gene delivery system significantly prolonged the survival of tumor bearing mice. Furthermore, mutants of bok show enhanced apoptotic activity, and the compositions and methods to utilize the same are included within the scope of the present invention. This provides realistic clinical applications for the present invention, such as the bok polynucleotide in gene therapy for ovarian cancer and other cancers.

The present invention generally relates to methods for inhibiting proliferation in a cancer cell and/or tumor cell, the method comprising contacting the cell with a Bok polypeptide in an amount effective to inhibit proliferation. The Bok polypeptide referred to herein may be a wild type or mutant form, so long as it has anti-cell proliferative, pro-apoptotic, and/or anti-tumor activity Inhibition of proliferation may be indicated by an induction of apoptosis of a cell, such as in cell culture, inhibition of growth of a cancer cell line, reduction in size of a tumor, and/or an increase in survivability. More preferably, in some embodiments the cell in which proliferation is to be inhibited is a cell in a living organism, for example a human. The inhibition of such transformation has great utility in the prevention and treatment of such transformation-driven events as cancer, tumorigenesis, and metastasis.

A Bok polypeptide may be contacted with or introduced to a cell through any of a variety of manners known to those of skill. The Bok polypeptide may be introduced through direct introduction of a Bok polypeptide to a cell. In this case, the Bok polypeptide may be obtained through any method known in the art, although it is expected that in vitro expression of the Bok polypeptide in a cell culture system may be a preferred manner of obtaining Bok.

Bok may also be introduced to a cell via the introduction of a polynucleotide that encodes the Bok polypeptide to the cell. For example, RNA or DNA encoding Bok may be introduced to the cell by any manner known in the art. In certain preferred embodiments, the Bok is introduced into the cell through the introduction of a DNA segment that encodes Bok. In some such embodiments, it is envisioned that the DNA segment further comprises the Bok gene (or Bok polynucleotide) operatively linked to its associated control sequences. For example, the bok gene may be operatively linked to a suitable promoter and a suitable terminator sequence. The construction of such gene/control sequence DNA constructs is well-known within the art. In particular embodiments, the promoter is selected from the group comprising of CMV, telomerase, TCF-4, or VEGF. In certain embodiments for introduction, the DNA segment may be located on a vector, for example, a plasmid vector or a viral vector. The virus vector may be, for example, selected from the group comprising retrovirus, adenovirus, herpesvirus, vaccina virus, and adeno-associated virus. Such a DNA segment may be used in a variety of methods related to the invention. The vector may be used to deliver a bok gene to a cell in one of the gene-therapy embodiments of the invention. Also, such vectors can be used to transform cultured cells, and such cultured cells could be used, inter alia, for the expression of Bok in vitro.

In particular embodiments the Bok is introduced into a cell that is a human cell. In many embodiments the cell is a tumor cell. In some embodiments, the cell overexpresses Bcl-2. In some presently preferred embodiments the tumor cell is a breast tumor cell, a prostrate tumor cell, or an ovarian tumor cell. However, Bok may be introduced into other tumor cells including, but not limited to, a bladder tumor cell, a testicular tumor cell, a colon tumor cell, a skin tumor cell, a lung tumor cell, a pancreatic tumor cell, a stomach tumor cell, an esophageal tumor cell, a brain tumor cell, a leukemia tumor cell, a liver tumor cell, an endometrial tumor cell, or a head and neck tumor cell. In some embodiments, the Bok composition is introduced by injection.

In some embodiments of the present invention, the inventor's discovery that Bok is able to inhibit proliferation will be used in combination with other anti-transformation/anti-cancer therapies. These other therapies may be known at the time of this application, or may become apparent after the date of this application. Bok may be used in combination with other therapeutic polypeptides, polynucleotides encoding other therapeutic polypeptides, or chemotherapeutic agents. For example, Bok may be used in conjunction with other known polypeptides, such as TNFα or p53. Bok may be used in conjunction with any suitable chemotherapeutic agent. In one representative embodiment, the chemotherapeutic agent is taxol. Bok also may be used in conjunction with radiotherapy. The type of ionizing radiation constituting the radiotherapy may be selected from the group comprising x-rays, γ-rays, and microwaves. In certain embodiments, the ionizing radiation may be delivered by external beam irradiation or by administration of a radionuclide. Bok also may be used with other gene-therapy regimes. In particular embodiments the Bok is introduced into a tumor. The tumor may be in an animal, in particular, a human. The Bok may be introduced by injection.

In some embodiments of the present invention, the inventor's discovery that Bok is able to inhibit tumor cell proliferation will be used in combination with other therapeutic agents. The other therapies may be known at the time of this application, or may become apparent after the date of this application. Bok may be used in combination with other therapeutic polypeptides, polynucleotides encoding other therapeutic polypeptides, chemotherapeutic agents, or radiotherapeutic agents. The Bok composition may be introduced into a tumor, and the tumor may be contained in an animal, in particular, a human. The Bok may be introduced by injection. In some embodiments, the other therapeutic agent induces apoptosis. In one preferred embodiment, the other agent capable of inducing apoptosis is TNFα. Other polypeptide inducers of apoptosis that may be used in combination with Bok include, but are not limited to, p53, Bax, Bak, Bcl-x, Bad, Bim, Bik, Bid, Harakiri, Ad E1B, Bad and ICE-CED3 proteases. In other embodiments, a chemotherapeutic agent capable of inducing apoptosis is used in combination with Bok. In one preferred embodiment, the chemotherapeutic agent capable of inducing apoptosis is taxol. In another embodiment, radiotherapy comprising ionizing radiation is the other apoptosis-inducing therapeutic agent. The type of ionizing radiation may be selected from the group comprising x-rays, γ-rays, and microwaves. The ionizing radiation may be delivered by external beam irradiation or by administration of a radionuclide.

The Bok gene products and polynucleotides of the present invention may also be introduced using any suitable method. A “suitable method” of introduction is one that places a bok gene product in a position to reduce the proliferation of a tumor cell. For example, injection, oral, and inhalation methods may be employed, with the skilled artisan being able to determine an appropriate method of introduction for a given circumstance. In some preferred embodiments, injection will be used. This injection may be intravenous, intraperitoneal, intramuscular, subcutaneous, intratumoral, intrapleural, or of any other appropriate form.

In certain other aspects of the present invention there are provided therapeutic kits comprising in a suitable container a pharmaceutical formulation of a Bok gene product or a polynucleotide encoding a Bok gene product. Such a kit may further comprise a pharmaceutical formulation of a therapeutic polypeptide, polynucleotide encoding a therapeutic polypeptide, and/or chemotherapeutic agent.

In a specific embodiment, there is a method of treating ovarian cancer, breast cancer, pancreatic cancer, or prostate cancer in an individual having the cancer, comprising contacting a cancer cell of the individual with a therapeutically effective amount of a polynucleotide encoding a Bok polypeptide, wherein the polynucleotide is comprised in a liposome.

The present invention provides a nuclear location for Bok, which was heretofore unknown. In some embodiments, the present invention provides a method of inhibiting cell proliferation, comprising the step of increasing nuclear levels of a Bok polypeptide. Exemplary methods to increase the levels of Bok in a nucleus includes rendering a nuclear export sequence in said Bok polypeptide ineffective. The term “rendering a nuclear export sequence ineffective” is herein defined as generating a Bok polypeptide that is retained in a nucleus for a longer period of time than a corresponding wild type Bok polypeptide.

Thus, the present invention is directed to the following embodiments:

In one embodiment of the present invention, there is a method of inhibiting cell proliferation comprising contacting a cell with a Bok polypeptide in an amount effective to inhibit the tumor cell proliferation.

In a further specific embodiment, the Bok-encoding polynucleotide further comprises at least one regulatory sequence. In a specific embodiment, the regulatory sequence is a promoter. In a further specific embodiment, the promoter is a CMV promoter. In an additional specific embodiment, the Bok-encoding polynucleotide is comprised in a vector. In another specific embodiment, the vector is a plasmid. In a further specific embodiment, the vector is a viral vector. In a specific embodiment, the viral vector is a retroviral vector, adenoviral vector, herpesviral vector, vaccinia viral vector, or adeno-associated viral vector. In a specific embodiment, the Bok-encoding polynucleotide is comprised with a nonviral gene delivery system, wherein the system comprises lipids, peptides, proteins, polymers, micelles, emulsion, or a combination thereof. In a further specific embodiment, the polynucleotide is complexed with the lipid. In a specific embodiment, the polynucleotide is comprised in a liposome. In a further specific embodiment, the cell is a tumor cell. In an additional specific embodiment, the tumor cell is an ovarian tumor cell, a breast tumor cell, a pancreatic cell, or a prostate tumor cell. In a further specific embodiment, the tumor cell is in a tumor.

In another specific embodiment, the method further comprises treating an individual having the tumor with surgery. In a specific embodiment, the tumor is in an animal. In another specific embodiment, the animal is a human. In an additional specific embodiment, the Bok polypeptide has tumor suppressor activity. In another specific embodiment, the tumor cell overexpresses a Bcl-2 polynucleotide. In one specific embodiment, the cell is p53 defective. In another specific embodiment, the cell is p53 wild type. In an additional specific embodiment, the cell is caspase-3 defective. In a further specific embodiment, the cell is caspase-3 wild type.

In a particular embodiment, the method further comprises treating the cell with a second agent, wherein the second agent is a therapeutic polypeptide, a polynucleotide encoding a therapeutic polypeptide, a chemotherapeutic agent, or a radiotherapeutic agent. In a specific embodiment, the method is further defined as comprising contacting the Bok polypeptide with the cell by injecting a Bok polynucleotide encoding the polypeptide into an animal comprising the cell. In a specific embodiment, the injection is intravenously, intradermally, intraarterially, intraperitoneally, intralesionally, intracranially, intraarticularly, intraprostaticaly, intrapleurally, intratracheally, intranasally, intravitreally, intravaginally, intratumorally, intramuscularly, subcutaneously, intravesicularly, mucosally, or intrapericardially. In a particular specific embodiment, the injection is intraperitoneally.

In an additional embodiment of the present invention, there is a method of treating a proliferative cell disorder in an individual comprising the step of administering to the individual a Bok composition in an amount effective to treat the disorder. In a specific embodiment, the Bok composition is a polypeptide, wherein the polypeptide is introduced into the cell by the direct introduction of the Bok polypeptide.

In a further specific embodiment, the polynucleotide is a deoxyribonucleic acid molecule. In an additional specific embodiment, the Bok-encoding polynucleotide further comprises at least one regulatory sequence. In an additional specific embodiment, the regulatory sequence is a promoter, such as a CMV promoter. In another specific embodiment, the Bok-encoding polynucleotide is comprised in a vector. In specific embodiments, the vector is a plasmid or is a viral vector. In a further specific embodiment, the viral vector is a retroviral vector, adenoviral vector, herpesviral vector, vaccinia viral vector, or adeno-associated viral vector. In a specific embodiment, Bok-encoding polynucleotide is comprised with a lipid or is complexed with a lipid. In a specific embodiment, the polynucleotide is comprised in a liposome. In a specific embodiment, the proliferative cell disorder is cancer. In a further specific embodiment, the cancer is ovarian cancer, breast cancer, or prostate cancer. In an additional specific embodiment, the method further comprises treating the cell with a second agent, wherein the second agent is a therapeutic polypeptide, a polynucleotide encoding a therapeutic polypeptide, a chemotherapeutic agent, or a radiotherapeutic agent. In a further specific embodiment, the method further comprises treating the cancer with surgery. In a specific embodiment, the cancer comprises a tumor cell that is p53 defective. In an additional specific embodiment, the cancer comprises a tumor cell that is p53 wild type. In another specific embodiment, the cancer comprises a tumor cell that is caspase-3 defective. IN an additional specific embodiment, the cancer comprises a tumor cell that is caspase-3 wild type. In a specific embodiment, the Bok polypeptide has tumor suppressor activity.

In a particular specific embodiment, the method is further defined as comprising contacting a cell with a polynucleotide encoding a Bok polypeptide by injecting the polynucleotide into the individual comprising the cell. In a further specific embodiment, the injection is intravenously, intradermally, intraarterially, intraperitoneally, intralesionally, intracranially, intraarticularly, intraprostaticaly, intrapleurally, intratracheally, intranasally, intravitreally, intravaginally, intratumorally, intramuscularly, subcutaneously, intravesicularly, mucosally, or intrapericardially. In another specific embodiment, the injection is intraperitoneally.

In another embodiment of the present invention, there is a kit for treatment of an individual with cancer, wherein the kit is housed in a suitable container, comprising a Bok composition; and a pharmaceutical carrier for the composition. In a specific embodiment, the Bok composition comprises a modified Bok polypeptide or polynucleotide encoding a modified Bok polypeptide. In another specific embodiment, the Bok composition is a Bok polypeptide. In a specific embodiment, the kit further comprises a therapeutic polypeptide, a polynucleotide encoding a therapeutic polypeptide, a chemotherapeutic agent or a radiotherapeutic agent.

In an additional specific embodiment, there is a method of treating ovarian cancer, breast cancer, pancreatic cancer, or prostate cancer in an individual having the cancer, comprising contacting a cancer cell of the individual with a therapeutically effective amount of a polynucleotide encoding a Bok polypeptide, wherein the polynucleotide is comprised in a liposome.

In an embodiment of the present invention, there is a method of inhibiting cell proliferation, comprising the step of providing to said cell a Bok composition comprising a defective nuclear export signal. In another embodiment of the present invention, there is a method of inhibiting cell proliferation, comprising the step of increasing nuclear levels of a Bok polypeptide. In a specific embodiment, the increasing step is further defined as rendering a nuclear export sequence in said Bok polypeptide ineffective.

In keeping with long-standing patent law convention, the words “a” and “an” when used in the present specification in concert with the word comprising, including the claims, denote “one or more.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B illustrate that rat bok nucleic acid sequence significantly induced apoptosis of human cancer cell lines. Human ovarian cancer cell lines PA1 (wild-type p53), 2774-10c (mutant p53), SKOV3 ip1 (p53null) (FIG. 1A) and human breast cancer cell lines MCF7 (wild-type p53, caspase-3 null), MDA-MB-468 (mutant p53, caspase-3 effective) (FIG. 1B) were transfected with liposomal rat bok plasmid (1-rbok)

FIGS. 2A, 2B, and 2C demonstrate that rat bok nucleic acid sequence carried by liposomes significantly prolonged the life of human ovarian cancer bearing mice. Female nude mice were inoculated with 2×10⁶ cells/mouse of human ovarian cancer cell lines PA1 (FIG. 2A), 2774-10c (FIG. 2B), and SKOV3-ip1 (FIG. 2C), separately. After 14 days, the mice were treated with multiple intraperitoneal injections of liposomal rat bok (L-rbok).

FIGS. 3A through 3C show that human bok nucleic acid sequence delivered by liposomes significantly inhibited the growth of human cancer cell lines. Human ovarian cancer cell lines (PA1, 2774-10c SKOV3 ip1) (FIG. 3A), human breast cancer cell lines (MCF7, MDA-MB-231, MDA-MB-435, and MDA-MB-468) (FIG. 3B) and human prostate cancer cell lines (DU145, PC3) (FIG. 3C) were tranfected with liposomal human bok plasmid for 5 hours in 10% serum containing medium, the dose was 2 μg DNA/10⁶ cells.

FIGS. 4A and 4B demonstrate that human bok nucleic acid sequence in liposomes significantly prolonged the life of mice bearing human ovarian cancer. Female nude mice were inoculated with 2×10⁶ cells/mouse of human ovarian cancer cell lines 2774-10c (FIG. 4A), and SKOV3-ip1 (FIG. 4B), separately. After 14 days, the mice were treated with multiple intraperitoneal injections of liposome carrying human bok plasmid (L-hbok).

FIGS. 5A and 5B show pro-apoptotic Bok is a potent inducer of cell death in the pancreatic cancer cell line PANC-1. In FIG. 5A, PANC-1 cells growing in a 2-chambered glass slide (1×10⁵ cells/chamber) were transiently transfected with the GFPexpressing AdTrack vector (pAdTrack) and the AdTrack vector containing cDNA inserts of wt hBok(pAdTrack-hBok). Transfected cells were analyzed for GFP expression by Fluorescent Microscopy 24 h after transfection. In FIG. 5B, the percentage of viable cells was determined as the number of GFP-expressing cells among the total cell population.

FIGS. 6A and 6B show the determination of the apoptotic potential of hBok in HEK 293T Cells. FIG. 6A demonstrates HEK293T cells (1×10⁵ cells/chamber) growing in two chambered slides that were transiently transfected with pcDNA3 (vector) and pcDNA3 expressing either rBok or hBok. Cells were fixed in 4% paraformaldehyde 24 h after transfection, and apoptosis was determined by the in vitro TUNEL assay. (apoptotic cells are indicated by an arrow). FIG. 6B shows that the NIH3T3 Fibroblast BCL-2 stable cell line and the NIH3T3 Neo cell line were seeded in a six well plate (3×10⁵ cells/well) and transiently transfected with pcDNA3 (vector), rBok, hBok and Bik. Cells were harvested 24 h and 48 h after transfection, and apoptosis was determined by FACS analysis

FIGS. 7A through 7D demonstrate the potential of hBok to kill breast cancer cells. Breast cancer cells were transiently co-transfected with: i.) pcDNA3 (control) and pLuc, and ii) phBok and pLuc. Cell viability was determined 24 h after transfection by the luciferase reporter assay (FIGS. 7A-7C, top panel). For the colony count assay, cells were transfected with vector and vector-expressing hBok. Forty eight hours after transfection, cells were split 1:20. The cells were then grown in a selection medium containing G418 (Genetisin) at 500 μg/mL for 2 weeks. G418-resistant colonies were stained with crystal violet/ethanol (1% to 20%) and counted (FIGS. 7A-7C bottom panel). FIG. 7D shows expression of Flag-tagged hBok that was confirmed by western blot using M5 anti-Flag antibody.

FIGS. 8A, 8B, and 8C illustrate the nuclear translocation of hBok. FIG. 8A shows an open reading frame of hBok showing the putative substrate motifs of protein kinase A (LRRSS), MAP kinase (DRSP), protein kinase C (TDK) and casein kinase (SAPE), and the putative NES (LLRLGDELE). FIG. 8B shows immunohistochemical analysis of Flag-tagged hBok. CHO cells and the breast cancer cell lines MDA-MB-231, MDA-MB-435, MCF-7 and MDA-MB-468 were transiently transfected with Flag-tagged hBok, fixed with 4% paraformaldehyde, and permeabilized with Triton X-100 16 to 18 h after transfection. hBok was detected with anti-Flag M5 polyclonal primary antibody and anti-rabbit secondary antibody conjugated with flourescein 5(6)-isothiocyanate. FIG. 8C indicates subcellular fractionation in HEK293T cells. The HEK293T cells were transiently transfected with either the pCDNA3 mammalian cell expression or the vector-expressing hBok. Cells were harvested 24 h after transfection and cytoplasmic and nuclear fractions obtained. Fifty μg of protein from each fraction was analyzed by western blot using the M5 anti-Flag monoclonal antibody.

FIGS. 9A and 9B show that hBok translocates to the nucleus. (FIG. 9A) CHO cells were seeded in a four chambered slide at a concentration of 5×10⁴ cells/chamber. The cells were transfected 24 h later with hBok using liposome as the delivery vehicle. Six hours after transfection, the treated group was incubated with LMB (10 ng/mL). Cells in both treated and untreated groups were harvested 12 h after transfection, fixed with 4% paraformaldehyde and permeabilized with Triton X-100. hBok was detected with anti-Flag M5 polyclonal primary antibody and anti-rabbit secondary antibody conjugated with flourescein 5(6)-isothiocyanate. (FIG. 9B) Comparison of nuclear hBok in transfected cell populations grown in either the absence or presence (10 ng/mL) of LMB.

FIGS. 10A through 10D show that the mutation of the putative NES of hBok sequesters the protein in the nucleus and enhances its killing potential. (FIG. 10A) The NES mutant of hBok was tested for its cell-killing ability. HEK293T cells were co-transfected with either wild type Bok or the NES mutant and pLuc and the luciferase assay carried out to determine the survival of the transfected cells. (FIG. 10B) Deletion of the putative NES of hBok sequesters it in the nucleus of CHO cells. The method used for transfecting and immunostaining the cells is the same as described in FIG. 9. (FIG. 10C) The NES mutant of hBok induces apoptosis at a faster rate than wild type. CHO cells (1×10⁵ cells/well) growing in a six well plate were transiently transfected with pcDNA3 (vector) and pcDNA3 expressing either hBok or hNESΔBok. Cells were fixed in cold 100% ethanol 24 h and 48 h after transfection. Apoptosis was determined by FACS analysis. (FIG. 10D). In a specific embodiment, one determines the onset of apoptosis in transfected cells by standard means in the art.

FIG. 11 demonstrates the NES mutant of hBok inhibits cell proliferation to a greater extent than wild type. The cDNA of wild type and NES-mutant hBok was cloned into the AdTrack vector such that both hBok and GFP could be independently expressed from the same expression vector. CHO cells and the breast cancer cell lines MDA-MB-435 and MDA-MB-231 were transiently transfected with either the vector or vector-expressing wild type or mutant hBok. The percentage of GFP-expressing cells was counted in three separate fields to determine the degree of cell death in each population.

### DETAILED DESCRIPTION OF THE INVENTION

I. The Present Invention

The present invention regards the pro-apoptotic bok polynucleotide as a tumor suppressor gene to treat human ovarian cancer, pancreatic cancer, breast cancer, prostate cancer, and other cancers. In some embodiments it is delivered by, for example, either a viral or non-viral delivery system into an appropriate recipient animal to suppress tumor growth and development. In one embodiment of the present invention the delivered Bok acts through an apoptosis mechanism to suppress tumor growth and development.

Thus, herein the inventors demonstrate that the bok gene exerted strong anti-tumor activity and behaved like a classic tumor suppressor. It was also shown that bok induces apoptosis through a p53-independent pathway; thus, the negative factors on the p53 pathway may have little or no effect on the antitumor activity of bok. In addition, the inventors have demonstrated that bok acts as a tumor suppressor gene, in that bok can kill caspase-3-null cancer cells. In the present invention, the therapeutic efficacy of bok polynucleotide-mediated gene therapy in the treatment of nude mice with human ovarian cancer xenografts was tested. The results indicated that administration of a bok polynucleotide to a mammal with cancer inhibits cancer growth and prolongs survival. In particular, intraperitoneal injections of liposome-bok encapsulated into a stabilized nonviral gene delivery system significantly inhibited the growth of orthotopic human ovarian cancers in the mice and prolonged the survival of the tumor-bearing mice. Furthermore, administration of a bok polynucleotide to pancreatic, prostate, and breast cell cancer lines resulted in decreased cell viability, likely through facilitating apoptotic mechanisms. In other embodiments, there is a Bok mutant comprising SEQ ID NO: 34. In particular, the present invention provides a Bok mutant comprising a defective nuclear export sequence.

Thus, the discovery of the antitumor activity of the bok gene significantly improves available methods and compositions for cancer gene therapy.

II. Definitions and Techniques Affecting Gene Products and Genes

A. Bok Gene Products and Genes

In this patent, the terms “Bok gene product” and “Bok” refer to proteins and polypeptides having amino acid sequences that are substantially identical to the native Bok amino acid sequences or which are biologically active. The term “biologically active” as used herein in some embodiments refers to capability of inducing apoptosis, having anti-tumor activity, cross-reacting with anti-Bok antibody raised against Bok, having anti-cellular proliferative activity, having enhanced nuclear localization, and the like. Thus, the Bok polynucleotides or polypeptides may be wild type for a particular organism, or they may be a mutant that comprises the afore-mentioned non-limiting exemplary activities. In specific embodiments, the mutant has enhanced nuclear localization compared to wild type. In an another embodiment, the mutant comprises a defective ubiquitination signal sequence, and/or any sequence which affects increasing nuclear localization of Bok to the nucleus, or decreases removal of Bok from the nucleus or destruction of Bok in the nucleus, cytoplasm, or both.

As described herein, hBok comprises the putative substrate motifs of protein kinase A (LRRSS), MAP kinase (DRSP), protein kinase C (TDK) and casein kinase (SAPE), and the putative NES (LLRLGDELE; SEQ ID NO: 37). In specific embodiments, one or more of these sites are mutated to inhibit their function. A skilled artisan recognizes based on teachings provided herein how to test the mutants for activity preferable for administration for inhibition of cell proliferation, and preferably an anti-tumor treatment. That is, exemplary activities tested include capability of inducing apoptosis, having anti-tumor activity, cross-reacting with anti-Bok antibody raised against Bok, having anti-cellular proliferative activity, enhanced nuclear localization. In some embodiments combination of mutations are utilized within one composition (having one or more constituent compounds), such as to inhibit cellular proliferation and/or administer in an effective amount to a tumor.

In specific embodiments, a Bok polynucleotide is mutated by methods well-known in the art and/or described herein to disrupt a MAP kinase recognition motif, a protein kinase C recognition motif, or both. In a specific embodiment, the MAP kinase mutation in a Bok polynucleotide occurs in the 5′-GCCTTTGACCGCTCGCCCACAGACAAG-3′ (SEQ ID NO: 38) wild type sequence. In specific embodiments, a MAP kinase sequence in Bok is mutated, such as in Bok (S21A) that comprises a polynucleotide comprised of 5′-GCCTTTGACCGCGCGCCCACAGACAAG-3′ (SEQ ID NO: 39) in which the serine residue of the MAPK kinase motif is substituted with alanine. In other embodiments, a protein kinase C motif is mutated in a Bok polynucleotide, such as comprising Bok (T23A) that comprises 5′-GACCGCTCGCCCGCGGACAAGGAGCTG-3′ (SEQ ID NO: 40) in which the threonine residue of the protein kinase C motif is substituted with alanine.

A skilled artisan recognizes that polynucleotide and polypeptide sequences are available in publicly available databases, such as the National Center for Biotechnology Information's GenBank database, or in commercially available databases such as from Celera Genomics, Inc. (Rockville, Md.). Examples of Bok amino acid sequences, followed by their GenBank Accession No., include: SEQ ID NO: 11 (NP_{—}115904.1); SEQ ID NO: 12 (NP_{—}055019.2); SEQ ID NO: 13 (NP_{—}059008.1); SEQ ID NO: 14 (AAF81282.1); SEQ ID NO: 15 (AAD51719.1); SEQ ID NO: 16 (AAG01182.1); SEQ ID NO: 17 (AAF25955.1); SEQ ID NO: 18 (AAF09129.1); SEQ ID NO: 19 (AAC61928.1); SEQ ID NO: 20 (AAB87418.1); SEQ ID NO: 24 (NP_{—}058058); SEQ ID NO: 25 (AAC53582); and/or SEQ ID NO: 26 (AAH06203). The term “Bok gene product” also includes analogs of Bok molecules that exhibit at least some biological activity in common with native Bok. Such analogs include, but are not limited to, truncated Bok polypeptides and Bok polypeptides having fewer amino acids than native Bok and/or mutants of Bok that retain pro-apoptotic activity, are anti-cell proliferative, or that have anti-tumor activity. Furthermore, those skilled in the art of mutagenesis will appreciate that homologs to the mouse Bok gene, including human homologs, which homologs are as yet undisclosed or undiscovered, may be used in the methods and compositions disclosed herein.

Bok occurs naturally in a long form (herein Bok-L), as exemplified by the amino acid sequences provided in SEQ ID NO: 27 and SEQ ID NO: 28, which are rat and human, respectively. A short form (herein Bok-S) also occurs naturally, as exemplified by SEQ ID NO: 29 and SEQ ID. NO: 30, in which there is a deletion leading to the fusion of the N-terminal half of the BH3 domain to the C-terminal half of the BH1 domain (herein, BOK-BH3_{inactive}).

In a preferred embodiment, the Bok composition and method of inhibiting cell proliferation or treating cancer using said composition comprises a defective nuclear export signal. In a specific embodiment, SEQ ID NO: 34 is utilized, in which the 71-LRL-73 amino acid residues within the putative nuclear export sequence (NES) are changed to 71-AAA-73.

The term “bok polynucleotide” or “bok nucleic acid” refers to any DNA sequence that is substantially identical to a DNA sequence encoding a Bok gene product as defined above. The term also refers to RNA, or antisense sequences compatible with such DNA sequences. A “Bok gene” may also comprise any combination of associated control sequences. The term “Bok polynucleotide” refers to any nucleic acid sequence that is substantially identical to a DNA sequence encoding a Bok gene product as defined above. The term also refers to RNA, or antisense sequences compatible with such DNA sequences. Examples of Bok nucleic acid sequences, followed by their GenBank Accession No. include: SEQ ID NO: 1 (NM_{—}032515); SEQ ID NO: 2 (NM_{—}014204); SEQ ID NO: 3 (NM_{—}017312); SEQ ID NO: 4 (AF275944); SEQ ID NO: 5 (AF174487); SEQ ID NO: 6 (AF290888); SEQ ID NO: 7 (AF216752); SEQ ID NO: 8 (AF089746); SEQ ID NO: 9 (AF051093); SEQ ID NO: 10 (AF027954); SEQ ID NO: 21 (NM_{—}016778); SEQ ID NO: 22 (AF027707); and/or SEQ ID NO: 23 (BC006203). A skilled artisan recognizes that there are multiple forms of known Bok polypeptides and further recognizes how to identify analogous specific residues upon comparing at least two similar sequences.

In a specific embodiment of the present invention, there is a polynucleotide that encodes a Bok gene product comprising a defective nuclear export sequence. In specific embodiments, the Bok polynucleotide sequence comprises CTGGCGGCGGCGGGCGAT (SEQ ID NO: 35).

Also included in the present invention are splice variants that encode long forms of the protein, as well as short forms having a truncation that deletes all or a part of the BH3 domain. A skilled artisan is aware that generally the long forms associate with anti-apoptotic proteins to form heterodimers, while the short forms induce cell killing without such heterodimerization. The Bok nucleic acid sequences may be naturally occurring or synthetic.

As used herein, the terms “Bok nucleic acid sequence,” “Bok polynucleotide,” and “Bok gene” refer to nucleic acids provided herein, homologs thereof, and sequences having substantial similarity and function. A skilled artisan recognizes that the sequences are within the scope of the present invention if they encode a product that has antitumor activity, pro-apoptotic activity, or has anti-cell proliferative activity, and furthermore knows how to obtain such sequences as is standard in the art.

The term “BH3_{inactive}”, or “BH3₁” is intended to generically refer to naturally occurring splice variants and synthetic variants of Bok in which deletions or amino acid substitutions made in the BH3 domain substantially inactivate or abrogate the heterodimerization activity of the protein. These variants may also be referred to as “channel only” proteins, because they retain the ability to form channels in the mitochondria that promote apoptosis. However, a skilled artisan recognizes that these variants are within the scope of the invention if they retain antitumor activity, as determined by methods provided herein and methods well known in the art. Examples are provided herein of BH3₁ variants, including but limited to: alanine substitutions at the highly conserved Bok glycine 75 residue, splice variants of Bok where there is a deletion of the amino acids 76-118; and a variant wherein a glycine substitution was made for leucine 71 to leucine 74 (BokGGGG: 71 LLRL 74 to 71 GGGG 74).

The BH3 domain has the consensus motif sequence: LRRAGDEFE.RYRR (SEQ ID NO: 31), and generally corresponds to the region of amino acids 71-82, in Bok (SEQ ID NO: 32, rat; and SEQ ID NO: 33, human).

The term “substantially identical”, when used to define either a Bok amino acid sequence or bok polynucleotide sequence, means that a particular subject sequence, for example, a mutant sequence, varies from the sequence of natural Bok by one or more substitutions, deletions, or additions, the net effect of which is to retain at least some biological activity of the Bok protein. Alternatively, DNA analog sequences are “substantially identical” to specific DNA sequences disclosed herein if: (a) the DNA analog sequence is derived from coding regions of the natural bok gene; or (b) the DNA analog sequence is capable of hybridization of DNA sequences of (a) under moderately stringent conditions and which encode biologically active Bok; or (c) DNA sequences which are degenerative as a result of the genetic code to the DNA analog sequences defined in (a) or (b). Substantially identical analog proteins will be greater than about 70%, 80%, 85%, 90%, 95%, or 100% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

1. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) which are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, (2) a penalty of 3.0 for each gap and an additional 0.01 penalty for each symbol and each gap; and (3) no penalty for end gaps.

2. Polynucleotide Sequences

In certain embodiments, the invention concerns the use of bok genes and gene products, such as the Bok that includes a sequence which is essentially that of the known bok gene, or the corresponding protein. The term “a sequence essentially as bok” means that the sequence substantially corresponds to a portion of the bok gene and has relatively few bases or amino acids (whether DNA or protein) which are not identical to those of Bok (or a biologically functional equivalent thereof, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of Bok will be sequences which are “essentially the same”.

In specific embodiments, there is a modified Bok polypeptide and/or a Bok polynucleotide encoding a modified Bok polypeptide. In the context of the present invention, the term “modified Bok polypeptide” or polynucleotide encoding a modified Bok polypeptide” relates to a polypeptide that has antitumor activity, pro-apoptotic activity, anti cell-proliferative activity, and/or nuclear-localizing activity that can be produced by one skilled in the art using the disclosure provided herein. After generating the modified Bok polypeptide or polynucleotide encoding the Bok polypeptide, one skilled in the art can test for its effectiveness using any teachings described herein for testing or methods known in the art.

In other embodiments there is an isolated Bok polypeptide comprising a defective nuclear export sequence, such as an isolated polypeptide comprising SEQ ID NO: 34. In additional embodiments, there is a method of inhibiting cell proliferation, comprising the step of providing to the cell a Bok composition comprising a defective nuclear export signal. An example for such a method includes the Bok composition comprised of SEQ ID NO: 34.

In a specific embodiment, the Bok polypeptide is introduced into the cell by the direct introduction of the Bok polypeptide. In another specific embodiment, the Bok polypeptide has a sequence of SEQ ID NO: 1, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 34. In an additional specific embodiment, the Bok polypeptide is introduced into the cell through the introduction of a Bok-encoding polynucleotide. In one specific embodiment, the polynucleotide encodes an amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 34. In an additional specific embodiment, the Bok polynucleotide has a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 21, SEQ ID NO: 22, or SEQ ID NO: 23. In another specific embodiment, the polynucleotide is a deoxyribonucleic acid molecule.

Bok genes that have functionally equivalent codons are also covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS.

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glutamic Acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood that amino acid and polynucleotide sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to polynucleotide sequences which may, for example, include various non-coding sequences flanking either of the 5′ or 3′ portions of the coding region or may include various internal sequences, i.e., introns, which are known to occur within genes.

In certain embodiments, the invention concerns the use of truncated Bok genes or polynucleotide sequences that encode a Bok polypeptide with less amino acids than native Bok. The present invention also encompasses the use of DNA segments which are complementary, or essentially complementary, to the sequences set forth in the specification. Polynucleotide sequences which are “complementary” are those which are capable of base-pairing according to the standard Watson-Crick complementarily rules. As used herein, the term “complementary sequences” means polynucleotide sequences which are substantially complementary, as may be assessed by the same nucleotide comparison set forth above, or as defined as being capable of hybridizing to the polynucleotide segment in question under relatively stringent conditions such as those described herein.

3. Biologically Functional Equivalents

As mentioned above, modification and changes may be made in the structure of Bok and still obtain a molecule having like or otherwise desirable characteristics. For example, certain amino acids may be substituted for other amino acids in a protein structure without appreciable loss of antitumor activity, pro-apoptotic activity, anti-cell proliferative activity, or a combination thereof. Since it is the interactive capacity and nature of a protein that defines that protein's biological functional activity, certain amino acid sequence substitutions and/or deletions can be made in a protein sequence (or, of course, its underlying DNA coding sequence) and nevertheless obtain a protein with like or even countervailing properties (e.g., antagonistic vs. agonistic). It is thus contemplated by the inventors that various changes may be made in the sequence of the Bok proteins or peptides (or underlying DNA) without appreciable loss of their biological utility or activity. Included in such changes are truncated Bok polypeptides and Bok polypeptides having less amino acid residues than native Bok.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged. This is the case in the present invention, where any changes in Bok that render the polypeptide incapable of suppressing transformation and inhibiting tumor cell proliferation would result in a loss of utility of the resulting peptide for the present invention.

Amino acid substitutions, such as those which might be employed in modifying Bok, are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those that are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

4. Sequence Modification Techniques

Modifications to the Bok peptides or polypeptides may be carried out using techniques such as site directed mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art as exemplified by publications (Adelman et al., 1983). As will be appreciated, the technique typically employs a phage vector that exists in both a single stranded and double stranded form. Typical vectors useful in site-directed mutagenesis include vectors such as the M13 phage (Messing et al., 1981). These phage are readily commercially available and their use is generally well known to those skilled in the art. Double stranded plasmids are also routinely employed in site-directed mutagenesis that eliminates the step of transferring the gene of interest from a plasmid to a phage.

In general, site-directed mutagenesis in accordance herewith is performed by first obtaining a single-stranded vector or melting apart the two strands of a double stranded vector which includes within its sequence a DNA sequence which encodes the Bok gene. An oligonucleotide primer bearing the desired mutated sequence is prepared, generally synthetically, for example by the method of Crea et al. (1978). This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected gene using site-directed mutagenesis is provided as a means of producing potentially useful Bok and is not meant to be limiting, as there are other ways in which sequence variants of these peptides may be obtained. For example, recombinant vectors encoding the desired genes may be treated with mutagenic agents to obtain sequence variants (see, e.g., a method described by Eichenlaub, 1979) for the mutagenesis of plasmid DNA using hydroxylamine.

5. Antisense Constructs

In some cases, mutant tumor suppressors may not be non-functional. Rather, they may have aberrant functions that cannot be overcome by replacement gene therapy, even where the “wild-type” molecule is expressed in amounts in excess of the mutant polypeptide. Antisense treatments are one way of addressing this situation. Antisense technology also may be used to “knock-out” function of Bok in the development of cell lines or transgenic mice for research, diagnostic and screening purposes.

Antisense methodology takes advantage of the fact that nucleic acids tend to pair with “complementary” sequences. By complementary, it is meant that polynucleotides are those that are capable of base-pairing according to the standard Watson-Crick complementarily rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarily to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

6. Synthetic Polypeptides

The present invention also describes Bok proteins and related peptides for use in various embodiments of the present invention. The Bok polypeptide may have fewer amino acids than native Bok. Relatively small peptides can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

7. Other Structural Equivalents

In addition to the Bok peptidyl compounds described herein, the inventors also contemplate that other sterically similar compounds may be formulated to mimic the key portions of the peptide structure. Such compounds may be used in the same manner as the peptides of the invention and hence are also functional equivalents. The generation of a structural functional equivalent may be achieved by the techniques of modeling and chemical design known to those of skill in the art. It will be understood that all such sterically similar constructs fall within the scope of the present invention.

B. Expression Vectors

In certain aspects of the present invention it may be necessary to express the Bok proteins and/or polypeptides. Throughout this application, the term “expression construct” is meant to include any type of genetic construct containing a polynucleotide coding for a gene product in which part or all of the polynucleotide encoding sequence is capable of being transcribed. The transcript may be translated into a protein, but it need not be. Thus, in certain embodiments, expression includes both transcription of a Bok gene and translation of a Bok mRNA into a Bok protein or polypeptide product. In other embodiments, expression only includes transcription of the polynucleotide encoding a Bok or its complement.

In order for the construct to effect expression of at least a Bok transcript, the polynucleotide encoding the Bok polynucleotide will be under the transcriptional control of a promoter. A “promoter” refers to a DNA sequence recognized by the synthetic machinery of the host cell, or introduced synthetic machinery, that is required to initiate the specific transcription of a gene. The phrase “under transcriptional control” means that the promoter is in the correct location in relation to the polynucleotide to control RNA polymerase initiation and expression of the polynucleotide.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete clement overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a Bok polynucleotide is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used to obtain high-level expression of the Bok polynucleotide. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosinase (melanoma), alpha-fetoprotein and albumin (liver tumors), CC10 (lung tumor) and prostate-specific antigen (prostate tumor) will permit tissue-specific expression of Bok polynucleotides. Table 2 lists several elements/promoters which may be employed, in the context of the present invention, to regulate the expression of Bok constructs. This list is not intended to be exhaustive of all the possible elements involved in the promotion of Bok expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a Bok construct. Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector.

TABLE 2
ENHANCER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
ENHANCER
α₁-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

Further, selection of a promoter that is regulated in response to specific physiologic signals can permit inducible expression of the Bok construct. For example, with the polynucleotide under the control of the human PAI-1 promoter, expression is inducible by tumor necrosis factor. Table 3 illustrates several promoter/inducer combinations:

TABLE 3

| Element | Inducer |
|---|---|
| MT II Phorbol Ester (TFA) | Heavy metals |
| MMTV (mouse mammary tumor virus) | Glucocorticoids |
| β-Interferon | Poly(rI)XPoly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease |
| | Virus |
| GRP78 Gene | A23187 |
| α-2-Macroglobulin | IL-6 |
| Vimentin | Serum |
| MHC Class I Gene H-2kB | Interferon |
| HSP70 Ela, SV40 Large T Antigen | |
| Proliferin | Phorbol Ester-TPA |
| Tumor Necrosis Factor | FMA |
| Thyroid Stimulating Hormone α Gene | Thyroid Hormone |

In certain embodiments of the invention, the delivery of an expression vector in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenicol acetyltransferase (CAT) (prokaryotic) may be employed. Immunologic markers also can be employed. The selectable marker employed is not believed to be important, so long as it is capable of being expressed along with the polynucleotide encoding Bok. Further examples of selectable markers are well known to one of skill in the art.

One typically will include a polyadenylation signal to effect proper polyadenylation of the transcript. The nature of the polyadenylation signal is not believed to be crucial to the successful practice of the invention, and any such sequence may be employed. The inventor has employed the SV40 polyadenylation signal in that it was convenient and known to function well in the target cells employed. Also contemplated as an element of the expression construct is a terminator. These elements can serve to enhance message levels and to minimize read through from the construct into other sequences.

The expression construct may comprise a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, expression vectors need not be viral but, instead, may be any plasmid, cosmid or phage construct that is capable of supporting expression of encoded genes in mammalian cells, such as pUC or BluescriptTM plasmid series.

C. Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules. In one approach, one would generate a three-dimensional structure for Bok or a fragment thereof. This could be accomplished by x-ray crystallography, computer modeling or by a combination of both approaches. An alternative approach, “alanine scan,” involves the random replacement of residues throughout molecule with alanine, and the resulting affect on function determined.

It also is possible to isolate a Bok specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved Bok activity or which act as stimulators, inhibitors, agonists, antagonists or Bok or molecules affected by Bok function. By use of cloned Bok sequences, sufficient amounts of Bok can be produced to perform crystallographic studies. In addition, knowledge of the polypeptide sequences permits computer employed predictions of structure-function relationships.

The present invention also contemplates the use of Bok and active fragments, and nucleic acids coding therefor, in the screening of compounds for activity in either stimulating Bok activity, overcoming the lack of Bok or blocking the effect of a mutant Bok molecule. In specific embodiments, the present invention provides methods of making one or a variety of Bok mutants, by means well-known in the art, that are screened to see if there is a desirable effect, according to activities described herein. For example, a candidate Bok mutant is screened for a pro-apoptotic activity, an anti-tumor activity, an anti-cell proliferative activity, and/or a nuclear retention activity (to a level greater than wild type).

The present invention also encompasses the use of various animal models. By developing or isolating mutant cells lines that fail to express normal Bok, one can generate cancer models in mice that will be highly predictive of cancers in humans and other mammals. These models may employ the orthotopic or systemic administration of tumor cells to mimic primary and/or metastatic cancers. Alternatively, one may induce cancers in animals by providing agents known to be responsible for certain events associated with malignant transformation and/or tumor progression. Finally, transgenic animals (discussed below) that lack a wild-type Bok may be utilized as models for cancer development and treatment.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

D. In vivo Delivery and Treatment Protocols

Where the gene itself is employed to introduce the gene products, a convenient method of introduction will be through the use of a recombinant vector which incorporates the desired gene, together with its associated control sequences. The preparation of recombinant vectors is well known to those of skill in the art and described in many references, such as, for example, Sambrook et al. (1989), specifically incorporated herein by reference.

In vectors, it is understood that the DNA coding sequences to be expressed, in this case those encoding the Bok gene products, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One may also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of the Bok will be preferred, there is no reason why other control sequences could not be employed, so long as they are compatible with the genotype of the cell being treated. Thus, one may mention other useful promoters by way of example, including, e.g., an SV40 early promoter, a long terminal repeat promoter from retrovirus, an actin promoter, a heat shock promoter, a metallothionein promoter, and the like.

For introduction of the Bok gene, it is proposed that one will desire to preferably employ a vector construct that will deliver the desired gene to the affected cells. This will, of course, generally require that the construct be delivered to the targeted tumor cells, for example, breast, genital, or lung tumor cells. It is proposed that this may be achieved most preferably by introduction of the desired gene through the use of a viral or non viral vectors to carry the Bok sequences to efficiently transfect the tumor, or pretumorous tissue. This infection may be achieved preferably by liposomal delivery but may also be via adenoviral, a retroviral, a vaccinia virus, herpesvirus or adeno-associated virus vector. These vectors have been successfully used to deliver desired sequences to cells and tend to have a high infection efficiency.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the Bgl I site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

1. Liposomal Transfection

Thus the expression construct comprising a polynucleotide encoding a Bok polypeptide may be entrapped in a liposome. Liposomes are structures created by mixing phospholipids with water, or hydration of phospholipid. The resultant bilayer structures tend to fold back upon themselves. Liposomes are frequently multilamellar, composed of concentric bilayer membranes separated by aqueous medium. They form spontaneously when phospholipids are suspended in an excess of aqueous solution The lipid components undergo self-rearrangement before the formation of closed structures and entrap water and dissolved solutes between the lipid bilayers (Ghosh and Bachhawat, 1991). Also contemplated are lipofectamine-DNA complexes.

The present invention also provides particularly useful methods for introducing Bok gene products into cells. One method of in vivo gene transfer which can lead to expression of genes transfected into cells involves the use of liposomes. Liposomes can be used for both in vitro and in vivo transfection. Liposome-mediated gene transfer seems to have great potential for certain in vivo applications in animals (Nicolau et al., 1987). Studies have shown that intravenously injected liposomes are taken up essentially in the liver and the spleen, by the macrophages of the reticuloendothelial system. The specific cellular sites of uptake of injected liposomes appears to be mainly spleen macrophages and liver Kupffer cells. Intravenous injection of liposomes/DNA complexes can lead to the uptake of DNA by these cellular sites, and result in the expression of a gene product encoded in the DNA (Nicolau, 1982).

The inventors contemplate that Bok gene products can be introduced into cells using liposome-mediated gene transfer. It is proposed that such constructs can be coupled with liposomes and directly introduced via a catheter, as described by Nabel et al. (1990). By employing these methods, Bok gene products can be expressed efficiently at a specific site in vivo, not just the liver and spleen cells which are accessible via intravenous injection. Therefore, this invention also encompasses compositions of DNA constructs encoding a Bok gene product formulated as a DNA/liposome complex and methods of using such constructs.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidyl-ethanolamine (DOPE), and/or 3b[N-(N′N′-dimethylaminoethane)-carbarmoyl cholesterol (DC-Chol), as well as other lipids known to those of skill in the art. Those of skill in the art will recognize that there are a variety of liposomal transfection techniques which will be useful in the present invention. Among these techniques are those described in Nicolau et al., 1987, Nabel et al., 1990, and Gao et al., 1991. In one embodiment of the present invention, liposomes comprising DC-Chol and DOPE which have been prepared following the teaching of Gao et al., 1991, are used. The inventors also anticipate utility for liposomes comprised of DOTMA, such as those which are available commercially under the trademark Lipofectin™, from Vical, Inc., in San Diego, Calif.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allows liposome-mediated transfer of DNA complex, for example, the liver and the spleen. In order to allow transfection of DNA into cells which are not accessible through intravenous injection, it is possible to directly inject the liposome-DNA complexes into a specific location in an animal's body. For example, Nabel et al. teach injection via a catheter into the arterial wall. In another example, the inventors have used intraperitoneal injection to allow for gene transfer into mice.

The present invention also contemplates compositions comprising a liposomal complex. This liposomal complex will comprise a lipid component and a DNA segment encoding a Bok gene.

The lipid employed to make the liposomal complex can be any of the above-discussed lipids. In particular, DOTMA, DOPE, and/or DC-Chol may form all or part of the liposomal complex. The inventors have had particular success with complexes comprising DC-Chol. In a preferred embodiment, the lipid will comprise DC-Chol and DOPE. While any ratio of DC-Chol to DOPE is expected to have utility, it is expected that those comprising a ratio of DC-Chol:DOPE between 1:20 and 20:1 will be particularly advantageous. The inventors have found that liposomes prepared from a ratio of DC-Chol:DOPE of about 1:10 to about 1:5 have been useful.

It is proposed that it will ultimately be preferable to employ the smallest region needed to suppress the Bok gene so that one is not introducing unnecessary DNA into cells which receive a Bok gene construct. Techniques well known to those of skill in the art, such as the use of restriction enzymes, will allow for the generation of small regions of Bok. The ability of these regions to inhibit tumor cell proliferation, tumorigenicity and transformation phenotype can easily be determined by the assays reported in the Examples.

In certain embodiments of the invention, the liposome may be complexed with a hemagglutinatin virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of polynucleotide in vitro and in vivo, then they are applicable for the present invention. Where a bacterial promoter is employed in the DNA construct, it also will be desirable to include within the liposome an appropriate bacterial polymerase.

2. Adenovirus

Another method for in vivo delivery involves the use of an adenovirus vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

Adenovirus is a particularly suitable gene transfer vector because of its midsized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, located at 16.8 mm is particularly efficient during the late phase of infection, and all the mRNA's issued from this promoter possess a 5′-tripartite leader (TL) sequence which makes them preferred mRNA's for translation.

In some cases, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

A particular method of introducing the Bok to an animal is to introduce a replication-deficient adenovirus containing the Bok gene. The replication-deficient construct made by E1B and E3 deletion also avoids the viral reproduction inside the cell and transfer to other cells and infection of other people, which means the viral infection activity is shut down after it infects the target cell. The Bok gene is still expressed inside the cells. Also, unlike retrovirus, which can only infect proliferating cells, adenovirus is able to transfer the Bok gene into both proliferating and non-proliferating cells. Further, the extrachromosomal location of adenovirus in the infected cells decreases the chance of cellular oncogene activation within the treated animal.

Introduction of the adenovirus containing the Bok gene product gene into a suitable host is typically done by injecting the virus contained in a buffer.

The nature of the adenovirus vector is not believed to be crucial to the successful practice of the invention. Of course, as discussed above, it is advantageous if the adenovirus vector is replication defective, or at least conditionally defective, The adenovirus may be of any of the 42 different known serotypes or subgroups A□F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 109-1011 plaque-forming units per ml, and they are highly infective. The life cycle of adenovirus does not require integration in to the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotatic inoculation into the brain (Le Gal La Salle et al., 1993).

3. Retroviruses

The retroviruses are a group of single-stranded RNA viruses characterized by an ability to convert their RNA to double-stranded DNA to infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene, termed y components is constructed (Mann et al., 1983). When a recombinant plasmid containing a human cDNA, together with the retroviral LTR and y sequences is introduced into this cell line (by calcium phosphate precipitation for example), the y sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. One limitation to the use of retrovirus vectors in vivo is the limited ability to produce retroviral vector titers greater than 106 infections U/mL. Titers 10- to 1,000-fold higher are necessary for many in vivo applications.

Several properties of the retrovirus have limited its use in lung cancer treatment (Stratford-Perricaudet and Perricaudet, 1991; (i) Infection by retrovirus depends on host cell division. In human cancer, very few mitotic cells can be found in tumor lesions. (ii) The integration of retrovirus into the host genome may cause adverse effects on target cells, because malignant cells are high in genetic instability. (iii) Retrovirus infection is often limited by a certain host range. (iv) Retrovirus has been associated with many malignancies in both mammals and vertebrates. (v) The titer of retrovirus, in general, is 100- to 1,000-fold lower than that of adenovirus.

4. Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpes viruses may be employed. They offer several attractive features for various mammalian cells (Friedmann, 1989; Ridgeway, 1988; Baichwal and Sugden, 1986; Coupar et al., 1988; Howrich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was cotransfected with wild□type virus into an avian hepatoma cell line. Cultures media containing high titers of the recombinant virus were used to infect primary duckling hepatocytes. Stable CAT gene expression was detected for at least 24 days after transfection (Chang et al., 1991).

5. Other Non-viral Vectors

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. As described above, delivery may be via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the polynucleotide encoding the gene of interest may be positioned and expressed at different sites. In certain embodiments, the polynucleotide encoding the gene may be stably maintained in the cell as a separate, episomal segment of DNA. Such polynucleotide segments or “episomes” encode sequences sufficient to permit maintenance and replication independent of or in synchronization with the host cell cycle. How the expression construct is delivered to a cell and where in the cell the polynucleotide remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty and Reshef (1986) also demonstrated that direct intraperitoneal injection of CaPO₄ precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs which can be employed to deliver a polynucleotide encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a polynucleotide encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a polynucleotide encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a polynucleotide into the cells, in vitro, and then the return of the modified cells back into an animal. This may involve the surgical removal of tissue/organs from an animal or the primary culture of cells and tissues. Anderson et al., U.S. Pat. No. 5,399,346, and incorporated herein in its entirety, disclose ex vivo therapeutic methods.

6. Protein Therapy

Another therapy approach is the provision, to a subject, of Bok polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

7. Lipid Compositions

In certain embodiments, the present invention concerns a novel composition comprising one or more lipids associated with at least one Bok polynucleotide or Bok polypeptide, protein, or peptide. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

a. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid, ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phoshoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phosholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

b. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

C. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

d. Lipid Composition Structures

In a preferred embodiment of the invention, the Bok composition may be associated with a lipid. A Bok composition associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/Bok composition associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-Bok composition or Superfect (Qiagen)-Bok composition complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10%to about 35% phosphatidyl choline, and about 1% of a drug. Thus, it is contemplated that lipid compositions of the present invention may comprise any of the lipids, lipid types or other components in any combination or percentage range.

e. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modern Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

f. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

g. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In certain less preferred embodiments, phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition or a liposome, because of the instability and leakiness of the resulting liposomes.

In particular embodiments, a Bok composition may be, for example, encapsulated in the aqueous interior of a liposome, interspersed within the lipid bilayer of a liposome, attached to a liposome via a linking molecule that is associated with both the liposome and the Bok composition, entrapped in a liposome, complexed with a liposome, etc.

h. Making Liposomes

A liposome used according to the present invention can be made by different methods, as would be known to one of ordinary skill in the art. Phospholipids can form a variety of structures other than liposomes when dispersed in water, depending on the molar ratio of lipid to water. At low ratios the liposome is the preferred structure.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the Bok composition, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the Bok composition is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/Bok composition or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs. Liposomal encapsulation has resulted in a lower toxicity and a longer serum half-life for such compounds (Gabizon et al., 1990).

Liposomes interact with cells to deliver agents via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

i. Liposome Targeting

Association of the Bok composition with a liposome may improve biodistribution and other properties of the Bok composition. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/Bok composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of Bok composition. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

j. Cross-Linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 4 details certain hetero-bifunctional cross-linkers considered useful in the present invention.

TABLE 4
HETERO-BIFUNCTIONAL CROSS-LINKERS

| | | | Spacer |
|---|---|---|---|
| | | | Arm |
| | | | Length\ |
| | | | after |
| | | | cross- |
| Linker | Reactive Toward | Advantages and Applications | linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC- | Primary amines | Extended spacer arm | 15.6 A |
| SPDP | Sulfhydryls | Water-soluble | |
| SMCC | Primary amines | Stable maleimide reactive | 11.6 A |
| | Sulfhydryls | group | |
| | | Enzyme-antibody conjugation | |
| | | Hapten-carrier protein | |
| | | conjugation | |
| Sulfo-SMCC | Primary amines | Stable maleimide reactive | 11.6 A |
| | Sulfhydryls | group | |
| | | Water-soluble | |
| | | Enzyme-antibody conjugation | |
| MBS | Primary amines | Enzyme-antibody conjugation | 9.9 A |
| | Sulfhydryls | Hapten-carrier protein | |
| | | conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo- | Primary amines | Hapten-Carrier conjugation | 0 |
| NHS | Carboxyl groups | | |
| ABH | Carbohydrates | Reacts with sugar groups | 11.9 A |
| | Nonselective | | |

In instances where a particular polypeptide does not contain a residue amenable for a given cross-linking reagent in its native sequence, conservative genetic or synthetic amino acid changes in the primary sequence can be utilized.

k. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a Bok composition may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific Bok composition delivery and/or targeting vehicle may comprise a specific binding ligand in combination with a liposome. The Bok composition to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and a Bok composition-binding agent. Others comprise a cell receptor-specific ligand to which Bok composition to be delivered has been operatively attached. For example, several ligands have been used for receptor-mediated gene transfer (Wu and Wu, 1987; Wagner et al., 1990; Perales et al., 1994; Myers, EPO 0273085), which establishes the operability of the technique. In another example, specific delivery in the context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

l. Liposome/Nucleic Acid Combinations

In certain embodiments, a liposome/Bok composition may comprise a nucleic acid, such as, for example, an oligonucleotide, a polynucleotide or a nucleic acid construct (e.g., an expression vector). Where a bacterial promoter is employed in the DNA construct that is to be transfected into eukaryotic cells, it also will be desirable to include within the liposome an appropriate bacterial polymerase.

It is contemplated that when the liposome/Bok composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4orf4 and the E. coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

m. Lipid Administration

The actual dosage amount of a lipid composition (e.g., a liposome-Bok composition) administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, subcutaneously, intravesicularlly, mucosally, intrapericardially, orally, topically, locally and/or using aerosol, injection, infusion, continuous infusion, localized perfusion bathing target cells directly or via a catheter and/or lavage.

E. Combined Therapy Protocols

Tumor cell resistance to anti-cancer agents represents a major problem in clinical oncology. The present invention may also be used in combination with conventional therapies to improve the efficacy of chemotherapy, radiotherapy, and/or surgery. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that Bok therapy could be used similarly in conjunction with chemotherapeutic, radiotherapeutic, or surgical intervention.

To kill cells, such as malignant or metastatic cells, using the methods and compositions of the present invention, one would generally contact a “target” cell with a Bok composition and at least one anti-cancer agent. These compositions would be provided in a combined amount effective to kill or inhibit proliferation of the cell. This process may involve contacting the cells with the Bok composition and the anti-cancer agent(s) or factor(s) at the same time. This may be achieved by contacting the cell with a single composition or pharmacological formulation that includes both agents, or by contacting the cell with two distinct compositions or formulations, at the same time, wherein one composition includes the Bok composition and the other includes the anti-cancer agent.

Alternatively, the Bok treatment may precede or follow the anti-cancer agent treatment by intervals ranging from min to weeks. In embodiments where the anti-cancer agent and Bok are applied separately to the cell, one would generally ensure that a significant period of time did not expire between the time of each delivery, such that the anti-cancer agent and Bok composition would still be able to exert an advantageously combined effect on the cell. In such instances, it is contemplated that one would contact the cell with both agents within about 6 h to one wk of each other and, more preferably, within about 24-72 h of each other, with a delay time of only about 48 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the Bok or the anti-cancer agent will be desired. Various combinations may be employed, where Bok is “A” and the anti-cancer agent is “B”:

A/B/A B/A/B B/B/A A/A/B B/B/B/A B/B/A/B

A/A/B/B A/B/A/B A/B/B/A B/B/A/A B/A/B/A B/A/A/B

A/A/A/B B/A/A/A A/B/A/A A/A/B/A A/B/B/B B/A/B/B

To achieve cell killing, both agents are delivered to a cell in a combined amount effective to kill the cell.

In one representative embodiment of the present invention, the anti-cancer agent is taxol (paclitaxel). This agent has proved has proved to be effective for the treatment of patients with metastatic breast or ovarian cancer, and has potential for patients with cervical or endometrial cancer. The regimen of paclitaxel administration has varied in clinical trials, the most common including a dosage of between 135 and 250 mg/m2 administered over an infusion period of 3 or 24 h once every 3 weeks (Wiseman and Spencer, 1998). Promising results have been achieved in phase I/II trials of a weekly regimen of paclitaxel (60 to 175 mg/m2). The objective response rate in patients with metastatic breast cancer (either pretreated or chemotherapy-naive) is generally between 20 and 35% with paclitaxel monotherapy, which compares well with that of other current treatment options including the anthracycline doxorubicin. Combination therapy with paclitaxel plus doxorubicin appears superior to treatment with either agent alone in terms of objective response rate and median duration of response (Wiseman and Spencer, 1998). The present invention contemplates the use of Bok combined with taxol and the use of Bok combined with taxol plus other anti-cancer agents such as doxorubicin.

Many anti-cancer agents are DNA damaging agents. DNA damaging agents or factors are defined herein as any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety chemotherapeutic agents function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. Many DNA damaging agents induce apoptosis. One aspect of the present invention is the use of Bok to sensitize tumor cells to apoptotic agents.

In treating cancer according to the invention, one would contact the tumor cells with a DNA damaging agent in addition to the Bok composition. This may be achieved by irradiating the localized tumor site with DNA damaging radiation such as X-rays, UV-light, γ-rays or even microwaves. Alternatively, the tumor cells may be contacted with the DNA damaging agent by administering to the subject a therapeutically effective amount of a pharmaceutical composition comprising a DNA damaging compound such as, adriamycin, 5-fluorouracil, etoposide, camptothecin, actinomycin-D, mitomycin C, or more preferably, cisplatin. The DNA damaging agent may be prepared and used as a combined therapeutic composition, or kit, by combining it with a Bok composition, as described above.

Agents that directly cross-link polynucleotides, specifically DNA, are envisaged and are shown herein, to eventuate DNA damage leading to a synergistic antineoplastic combination. Agents such as cisplatin, and other DNA alkylating may be used. Cisplatin has been widely used to treat cancer, with efficacious doses used in clinical applications of 20 mg/m2 for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m2 at 21 day intervals for adriamycin, to 35-50 mg/m2 for etoposide intravenously or double the intravenous dose orally.

Agents that disrupt the synthesis and fidelity of polynucleotide precursors and subunits also lead to DNA damage. As such a number of polynucleotide precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of DNA damage, or the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventor proposes that the regional delivery of Bok compositions to patients with tumors will be a very efficient method for delivering a therapeutically effective gene to counteract the clinical disease. Similarly, the chemotherapy, radiotherapy, or surgery may be directed to a particular, affected region of the subject's body. Alternatively, systemic delivery of the Bok or the DNA damaging agent may be appropriate in certain circumstances, for example, where extensive metastasis has occurred.

Cytokine therapy also has proven to be an effective partner for combined therapeutic regimens. Various cytokines may be employed in such combined approaches. Examples of cytokines include IL-1a IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNFa, TNFβ, LAF, TCGF, BCGF, TRF, BAF, BDG, MP, LIF, OSM, TMF, PDGF, IFN-a, IFN-β, IFN-γ. Cytokines are administered according to standard regimens, as described below, consistent with clinical indications such as the condition of the patient and relative toxicity of the cytokine.

A number of polypeptides are known to induce apoptosis and may be used in the combination therapies of the present invention. In one embodiment, the combination therapy is the use of Bok with a polypeptide form the tumor necrosis factor (“TNF”) family. In a preferred embodiment, the TNF polypeptide is TNFα. Other polypeptide inducers of apoptosis that may be used in the present invention include, but are not limited to, p53, Bax, Bak, Bcl-x, Bad, Bim, Bik, Bid, Harakiri, Ad E1B, Bad and ICE-CED3 proteases.

F. Pharmaceutical Compositions and Routes of Administration

Bok compositions of the present invention will have an effective amount of a gene for therapeutic administration in combination with an effective amount of a compound (second agent) that is an anti-cancer agent as exemplified above. Such compositions will generally be dissolved or dispersed in a pharmaceutically acceptable carrier or aqueous medium. The term “effective” as used herein refers to providing inhibition of proliferation of at least one cell, such as in a human; providing retardation of growth of a tumor; providing shrinking in size or eradication of a tumor; providing impeding metastases; and/or providing amelioration of a cancer symptom, and so forth.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The vectors of the present invention are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for inhibiting tumor cell proliferation may be assembled together in a kit. When the components of the kit are provided in one or more liquid solutions, the liquid solution preferably is an aqueous solution, with a sterile aqueous solution being particularly preferred.

For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of cancerous tissues may be accomplished in any one of a variety of ways. Plasmid vectors and retroviral vectors, adenovirus vectors, and other viral vectors all present means by which to target human cancers. The inventors anticipate particular success for the use of liposomes to target Bok genes to cancer cells. For example, DNA encoding Bok may be complexed with liposomes in the manner described above, and this DNA/liposome complex injected into patients with certain forms of cancer, such as breast cancer, intravenous injection can be used to direct the gene to all cell. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course, the potential for liposomes that are selectively taken up by a population of cancerous cells exists, and such liposomes will also be useful for targeting the gene.

Those of skill in the art will recognize that the best treatment regimens for using Bok to suppress tumors can be straightforwardly determined. This is not a question of experimentation, but rather one of optimization, which is routinely conducted in the medical arts. The in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of Bok used in mice. In certain embodiments it is envisioned that the dosage may vary from between about 1 mg Bok DNA/Kg body weight to about 5000 mg Bok DNA/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg Bok DNA/Kg body to about 20 mg Bok DNA/ Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

G. Transgenic Animals/Knockout Animals

In one embodiment of the invention, transgenic animals are produced which contain a functional transgene encoding a functional Bok polypeptide or variants thereof. Transgenic animals expressing Bok transgenes, recombinant cell lines derived from such animals and transgenic embryos may be useful in methods for screening for and identifying agents that induce or repress function of Bok. Transgenic animals of the present invention also can be used as models for studying indications such as cancers.

In one embodiment of the invention, a Bok transgene is introduced into a non-human host to produce a transgenic animal expressing a human or murine Bok gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous Bok by homologous recombination between the transgene and the endogenous gene; or the endogenous gene may be eliminated by deletion as in the preparation of “knock-out” animals. Typically, a Bok gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress Bok or express a mutant form of the polypeptide. Alternatively, the absence of a Bok in “knock-out” mice permits the study of the effects that loss of Bok protein has on a cell in vivo. Knock-out mice also provide a model for the development of Bok-related cancers.

As noted above, transgenic animals and cell lines derived from such animals may find use in certain testing experiments. In this regard, transgenic animals and cell lines capable of expressing wild-type or mutant Bok may be exposed to test substances. These test substances can be screened for the ability to enhance wild-type Bok expression and or function or impair the expression or function of mutant Bok.

III. Apoptosis-Inducing Proteins

Strictly regulated cell death is required for the development of multilineage organisms and the maintenance of homeostasis within tissues. Differentiation status of an individual cell directly affects whether it can execute a suicidal response following a death stimulus varies. Both positive and negative regulators of programmed cell death (apoptosis) have been identified. Bcl-2 is a repressor of programmed cell death (Vaux et al., 1988), and recently, other Bcl-2 homologues were shown to inhibit apoptosis. However, one homolog of Bcl-2, Bax, mediates an opposite effect through acceleration of apoptosis. In the Bcl-2 family there is notable homology clustered within two conserved regions: Bcl-2 homology domains 1 and 2 (BH1 and BH2) (Oltvai et al., 1993; Boise et al., 1993; Kozopas et al., 1993; Lin et al., 1993). Members of the Bc1 family include Bax, Bcl-X_{L}, Mcl-1, A1 and several open reading frames in DNA viruses. Another conserved domain in Bax, distinct from BH1 and BH2, is termed BH3 and mediates cell death and protein binding functions (Chittenden et al., 1995). A subset of the pro-apoptotic proteins contains only the BH3 domain, implying that this particular domain may be uniquely important in the promotion of apoptosis (Diaz et al., 1997).

In vivo Bax homodimerizes and also forms heterodimers with BCL-2, and overexpressed Bax overrides the death repressor activity of BCL-2 (Oltvai et al., 1993). Bax expression levels higher than Bcl-2 expression levels in bladder tumors correlates to an improved patient prognosis. In patients whose tumors expressed more Bcl-2 than Bax mRNA, early relapses were much more frequently observed (Gazzaniga et al., 1996).

Recently it was reported that a splice variant of Bax, Bax-alpha, was expressed in high amount in normal breast epithelium, whereas only weak or no expression was detected in 39 out of 40 cancer tissue samples examined (Bargou et al., 1996), and downregulation of Bax-alpha was detected in different histological subtypes. Furthermore, when Bax-alpha was transfected into breast cancer cell lines under the control of a tetracycline-dependent expression system, Bax restored sensitivity of the cancer cells toward both serum starvation and APO-I/Fas-triggered apoptosis, significantly reducing tumor growth in SCID mice. Therefore, it was proposed that disruption of apoptosis pathway may contribute to the pathogenesis of breast cancer at least in part due to an imbalance between members of the Bcl-2 gene family (Bargou et al., 1996).

Additional members of the Bcl-2 family of apoptosis-inducing proteins have been identified. Bak, a new member of the Bcl-2 family, is expressed in a wide variety of cell types and binds to the Bcl-2 homologue Bcl-x2 in yeast (Farrow et al., 1995; Chittenden et al., 1995). A domain in Bak was identified as both necessary and sufficient for cytotoxicity activity and binding to Bcl-x1. Furthermore, sequences similar to this domain that are distinct from BH1 and BH2 have been identified in Bax and Bipl. This domain is critical for mediating the function of multiple cell death-regulatory proteins that interact with Bcl-2 family members (Chittenden et al., 1995).

Overexpression of Bak in sympathetic neurons deprived of nerve growth factor accelerated apoptosis and blocked the protective effect of co-injected E1B 19K. The adenovirus E1B 19K protein is known to inhibit apoptosis induced by E1A, tumor-necrosis factor-alpha, FAS antigen and nerve growth factor deprivation (Farrow et al., 1995). Expression of Bak induced rapid and extensive apoptosis of serum-deprived fibroblasts, which suggests that Bak is directly involved in activating the cell death machinery (Chittenden et al., 1995). In the normal and neoplastic colon, mucosal expression of immunoreactive Bak co-localized with sites of epithelial cell apoptosis. Induction of apoptosis in the human colon cancer cell line HT29 and the rat normal small intestinal cell line 1EC 18 in culture was accompanied by increased Bak expression without consistent changes in expression of other Bcl-2 homologous proteins (Mos et al., 1996). Therefore, Bak was also suggested to be the endogenous Bcl-2 family member best correlated with intestinal cell apoptosis (Moss et al., 1996).

Unlike Bax, however, Bak can inhibit cell death in an Epstein-Barr-virus-transformed cell line. Tissues with unique distribution of Bak messenger RNA include those containing long-lived, terminally differentiated cell types (Krajewski, et al., 1996), suggesting that cell-death-inducing activity is broadly distributed, and that tissue-specific modulation of apoptosis is controlled primarily by regulation of molecules that inhibit apoptosis (Kiefer et al., 1995).

Another member of the Bcl-2 family, Bad, possesses the key amino acid motifs of BH1 and BH2 domains. Bad lacks the classical C-terminal signal-anchor sequence responsible for the integral membrane positions of other family members. Bad selectively dimerizes with Bcl-x_{L} as well as Bcl-2, but not with Bax, Bcl-Xs-Mcl1, A1 or itself. Bad reverses the death repressor activity of Bcl-X_{L}, but not that of Bcl-2 (Yang et al., 1995; Ottilie et al., 1997; Zha et al., 1997).

Bik, another member of the Bcl-2 family, interacts with the cellular survival-promoting proteins, Bcl-2 and Bcl-X_{L} as well as the viral survival-promoting proteins, Epstein Barr virus-BHRF1 and adenovirus E1B-19 kDa. In transient transfection assays, Bik promotes cell death in a manner similar to Bax and Bak, other pro-apoptotic members of the Bcl-2 family. This pro-apoptosis activity of Bik can be suppressed by coexpression of Bcl-2, Bcl-X_{L}, EBV-BHRF1 and E1B-19 kDa proteins, which suggests that Bik may be a common target for both cellular and viral anti-apoptotic proteins. While Bik does not contain overt homology to the BH1 and BH2 conserved domains characteristic of the Bcl-2 family, it shares a 9 amino acid domain (BH3) with Bax and Bak, which may be a critical determinant for the death-promoting activity of these proteins (Boyd et al., 1995; Han et al., 1996).

The Bcl-2 family is composed of various pairs of antagonist and agonist proteins that regulate apoptosis, although whether their function is interdependent remains unclear. Utilizing gain-and loss of- function models of Bcl-2 and Bax, Knudson et al. (1997), demonstrated that apoptosis and thymic hypoplasia, characteristic of Bcl-2-deficient mice, are largely absent in mice also deficient in Bax. A single copy of Bax promoted apoptosis in the absence of Bcl-2. However, overexpression of Bcl-2 still repressed apoptosis in the absence of Bax. While an in vivo competition exists between Bax and Bcl-2, each is able to regulate apoptosis independently. Bax has been shown to form channels in lipid membranes and trigger the release of liposome-encapsulated carboxyluorescein at both neutral and acidic pH. At physiological pH, release could be blocked by Bcl-2. In planer lipid bilayers, Bax formed pH- and voltage-dependent ion-conduction channels. Thus, the pro-apoptotic effects of Bax may be elicited through an intrinsic pore-forming activity that can be antagonized by Bcl-2 (Antonsson et al., 1997). Two other members of this family, Bcl-2 and Bcl-1, were also shown to form pores in lipid membranes (Schendel et al., 1997).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Bok Induced Apoptosis Occurred in Caspase-3 Defective Cells and was Independent of p53 Status

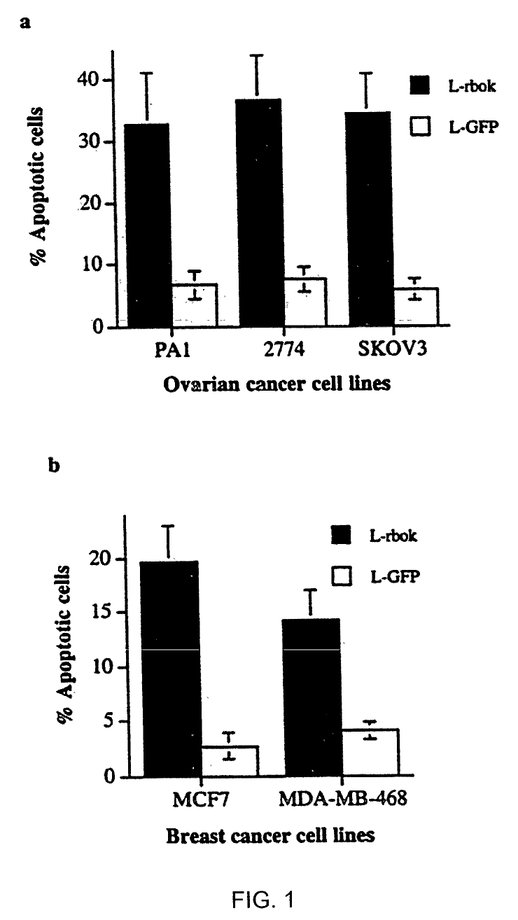

The bok cDNA was subcloned into the mammalian expression vector driven by cytomegalovirus (CMV) promoter. Various human ovarian and breast cancer cell lines of different p53 and caspase-3 status were transfected with SN carried bok plasmid (SN-bok). One day after transfection, the apoptotic cells were assessed by detection of the PI-stained DNA in representative cells by flow cytometry. The results in FIG. 1 indicate that the transfected bok gene significantly induced apoptosis of human ovarian and breast cancer cell lines, and that the apoptotic function is independent of p53 and caspase-3. In FIG. 1, human ovarian cancer cell lines PA1 (wild-type p53), 2774-10c (mutant p53), SKOV3 ip1 (p53 null) (FIG. 1A) and human breast cancer cell lines MCF7 (wild-type p53, caspase-3 null), MDA-MB-468 (mutant p53, caspase-3 effective) (FIG. 1B) were transfected with liposomal rat bok plasmid (l-rbok) for 5 hours in 10% serum-containing medium, the dose was 2 μg DNA/10⁶ cells. Liposomal green fluorescence gene (L-GFP) transfected cells were used as control. One day later, the cells were harvested, fixed with 70% ethanol, and stained with propidium iodide. The apoptotic cells were determined with standard FACS assay using flow cytometry. The results represent the mean ±SD from three independent experiments.

### Example 2

### Study of the Therapeutic Function of CMV-Bok in Ovarian Cancer Models

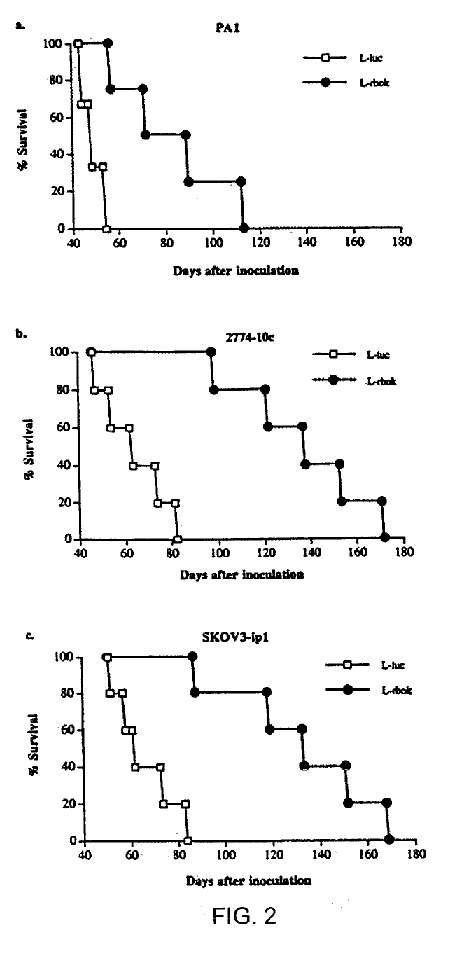

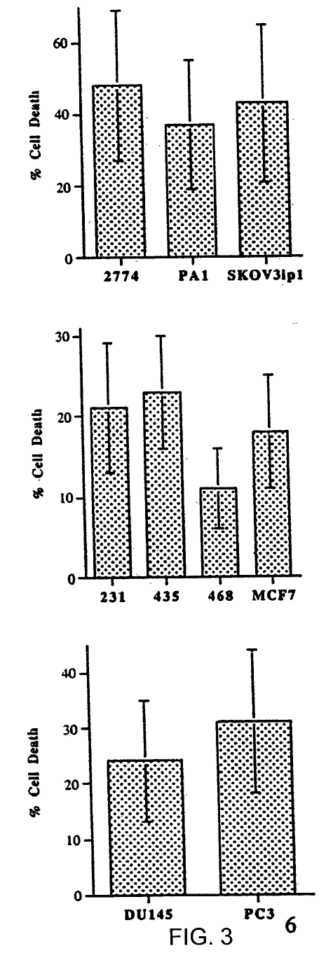

It was tested whether the CMV-driven bok can inhibit human ovarian cancer xenografts in mice. The human ovarian cancer cell lines PA1 (FIG. 2A), 2774-10c (FIG. 2B), and SKOV3-ip1 (FIG. 2C) were inoculated intraperitoneally into female nude mice. This model mimicked human ovarian cancer metastases in peritoneal cavity. Two weeks after tumor inoculation, the SN-bok was injected into the peritoneal cavity of the mice.

FIGS. 2A, 2B, and 2C show Rat bok carried by liposomes significantly prolonged the life of human ovarian cancer bearing mice. Female nude mice were inoculated with 2×10⁶ cells/mouse of human ovarian cancer cell lines PA1 (FIG. 2A), 2774-10c (FIG. 2B), and SKOV3-ip1 (FIG. 2C), separately. After 14 days, the mice were treated with multiple intraperitoneal injections of liposomal rat bok (L-rbok). The mice were subjected to 10 injections administered once every 3 days. Each dose was 30 μg DNA/mouse. The control mice were injected with the same dose of liposomal luciferase plasmid (L-luc). The life span of the mice was recorded. The data presented in FIG. 2 was obtained from groups of 3 to 5 mice.

FIG. 3 shows human bok gene delivered by liposomes significantly inhibited the growth of human cancer cell lines. Human ovarian cancer cell lines (PA1, 2774-10c SKOV3 ip1) (FIG. 3A), human breast cancer cell lines (MCF7, MDA-MB-231, MDA-MB-435, and MDA-MB-468) (FIG. 3B) and human prostate cancer cell lines (DU145, PC3) (FIG. 3C) were tranfected with liposomal human bok plasmid for 5 hours in 10% serum containing medium, the dose was 2 μg DNA/10⁶ cells. Non transfected cells were used as control. One day later, the cells were harvested and stained with trypan blue. The viable cells were counted. The results (% Cell Death=1-number of transfected cells/number of non transfected cells) represent the mean ±SD from three independent experiments.

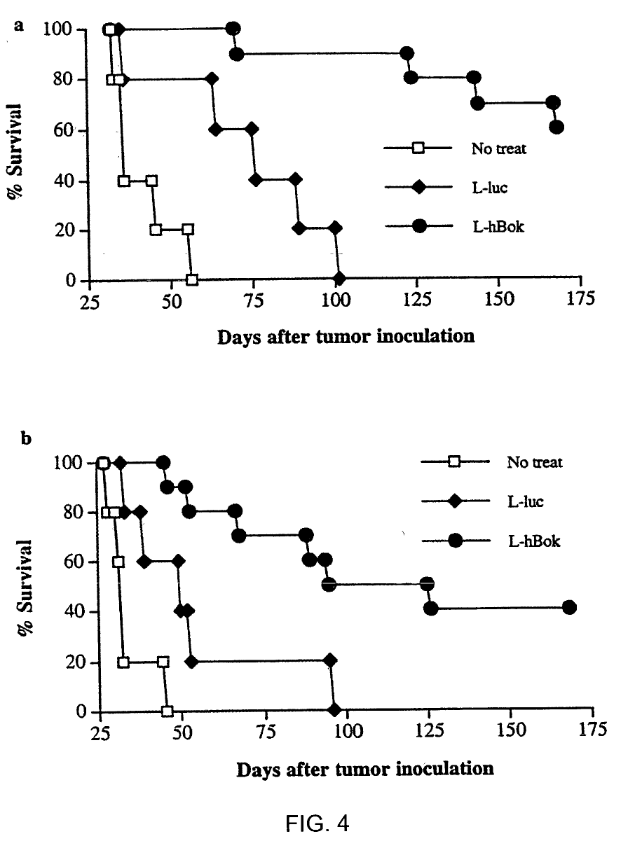

FIG. 4 shows that human bok significantly prolonged the life of mice bearing human ovarian cancer. Female nude mice were inoculated with 2×10⁶ cells/mouse of human ovarian cancer cell lines 2774-10c (FIG. 4A), and SKOV3-ip1 (FIG. 4B), separately. After 14 days, the mice were treated with multiple intraperitoneal injections of liposome carrying human bok plasmid (L-hbok). The mice were subjected to 10 injections administered once every 3 days. Each dose was 20 μg DNA/mouse. The control mice received injections of the same dose of the liposome-luciferase plasmid (L-luc) or no treatment (No treat). The life span of the mice was recorded. The data presented was obtained from groups of 5 to 10 mice.

Thus, the results presented in FIGS. 2, 3, and 4 showed that bok significantly inhibited the tumor growth and prolonged the survival of the tumor-bearing mice.

### Example 3

### Bok and Pancreatic Cancer Therapy

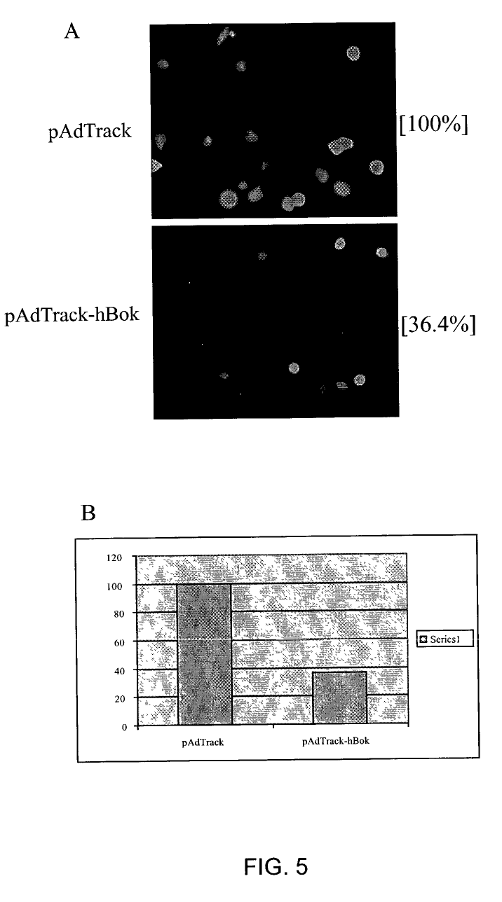

A skilled artisan recognizes that Bok, in specific embodiments, is useful in a variety of cancers. The present invention demonstrates pro-apoptotic Bok is a potent inducer of cell death in the pancreatic cancer cell line PANC-1. In FIG. 5A, PANC-1 cells growing in a 2-chambered glass slide (1×10⁵ cells/chamber) were transiently transfected with the GFPexpressing AdTrack vector (pAdTrack) and the AdTrack vector containing cDNA inserts of wt hBok (pAdTrack-hBok). Transfected cells were analyzed for GFP expression by fluorescent microscopy. FIG. 5B demonstrates the cells 24 h after transfection. The percentage of viable cells was determined as the number of GFP expressing cells among the total cell population.

Thus, pro-apoptotic Bok is a useful therapy for cancers other than ovarian, such as pancreatic. A skilled artisan recognizes that in similar embodiments, prostate and breast cancers are treated.

### Example 4

### Human Bok Induces Apoptosis in HEK 293T Cells

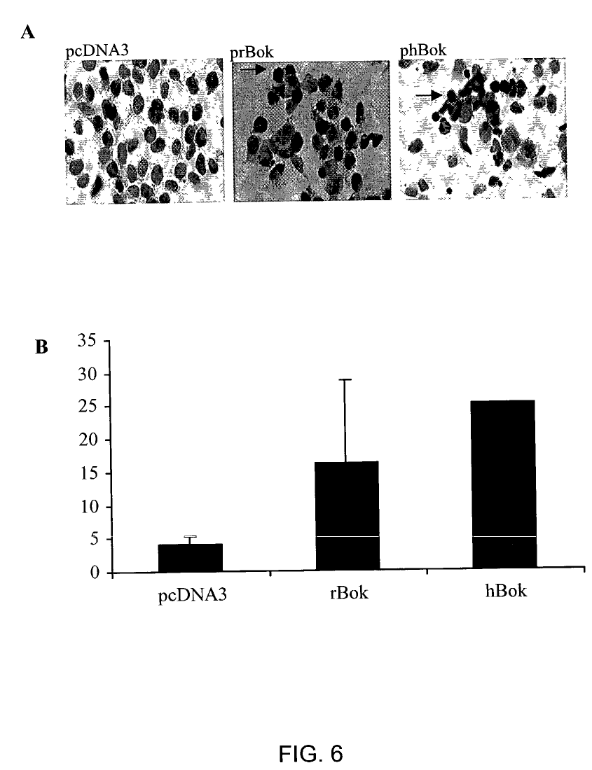

Sequence analysis of the human homolog of the rBok gene showed an almost 95% identity to rBok. However, hBok differs from rBok by nine amino acid residues randomly distributed throughout the protein. rBok induces apoptosis in mammalian cells (Hsu et al., 1997; Inohara et al., 1998). To determine if the apoptotic activity of hBok was similar to that of rBok, the pcDNA3 empty-vector and vector-expressing either hBok or rBok were transiently transfected into HEK293T cells. HEK293T transfectants were harvested 24 h after transfection and the occurrence of apoptosis determined by TUNEL assay (FIG. 6). Thirty percent of hBok HEK293T transfectants underwent apoptosis, while 20% of rBok transfectants and 5% of vector transfectants underwent apoptosis. Thus, hBok is as effective as rBok in its proapoptotic activity. The proapoptotic activity of rBok is not antagonized by Bcl-2.

Further tests are performed to compare the similarity between rBok and hBok activity. For example, the effect of Bcl-2 on hBok proapoptotic activity is characterized.

### Example 5

### Human Bok Inhibits the Growth of Breast Cancer Cells

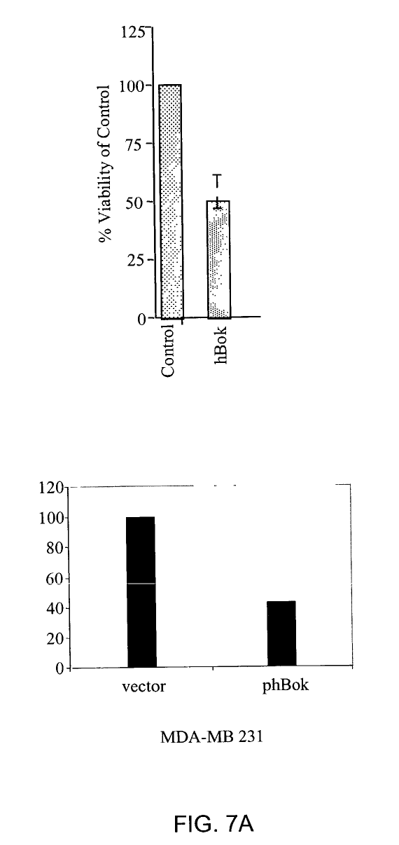

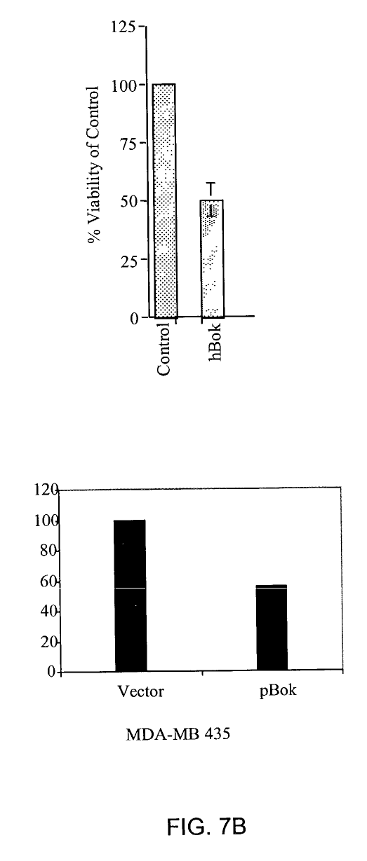

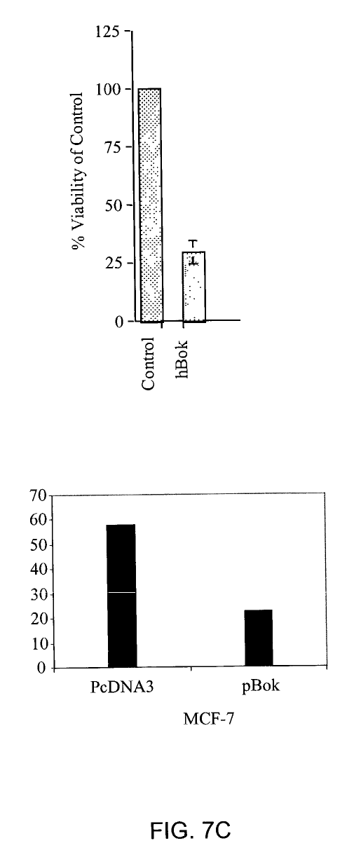

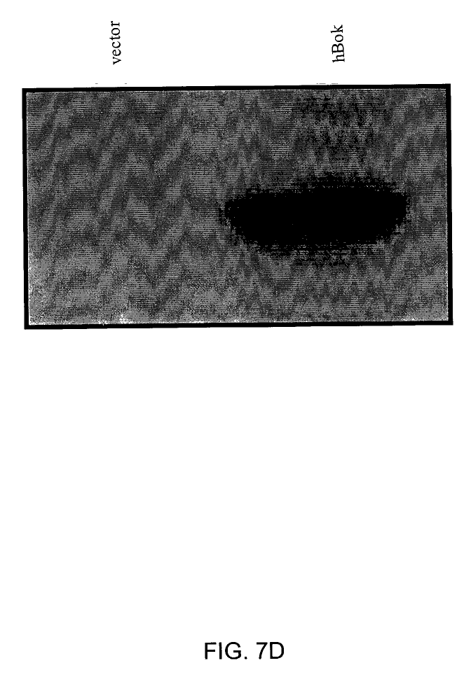

Having shown that hBok is a potent inducer of apoptosis, its ability to kill the breast cancer cell lines MCF-7, MDA-MB 231 and MDA-MB 435 in tissue culture was determined (FIGS. 7A, 7B and 7C). The luciferase reporter assay and the colony forming assay were carried out to determine the killing potential of hBok. The viability of the transfected breast cancer cells was assessed 24 h after transfection by determining the level of luciferase expression. hBok induced cell death in each of the breast cancer cell lines (FIGS. 7A, 7B, and 7C, top panels). The colony formation assay further demonstrated that hBok inhibits proliferation of breast cancer cells (FIGS. 7A, 7B, and 7C, bottom panels). The results of these two assays confirm that the proapoptotic activity of hBok was specific, because transfection of the vector had minimal effect on cell survival.

### Example 6

### hBok Translocates to the Nucleus

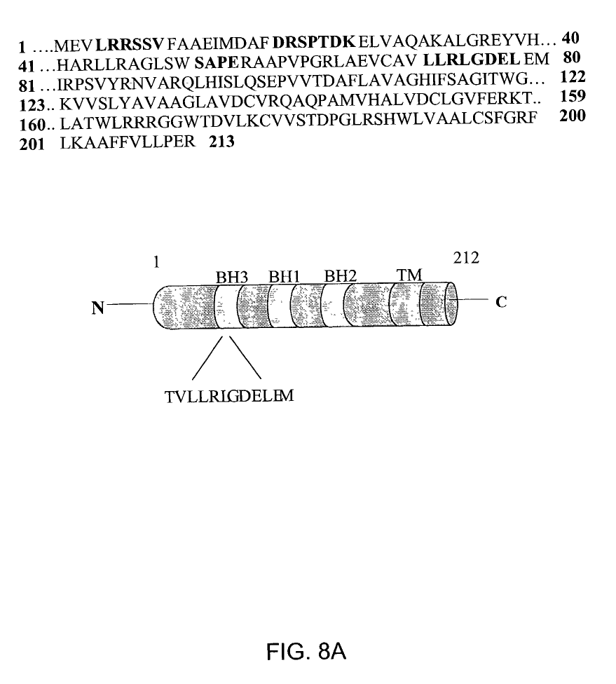

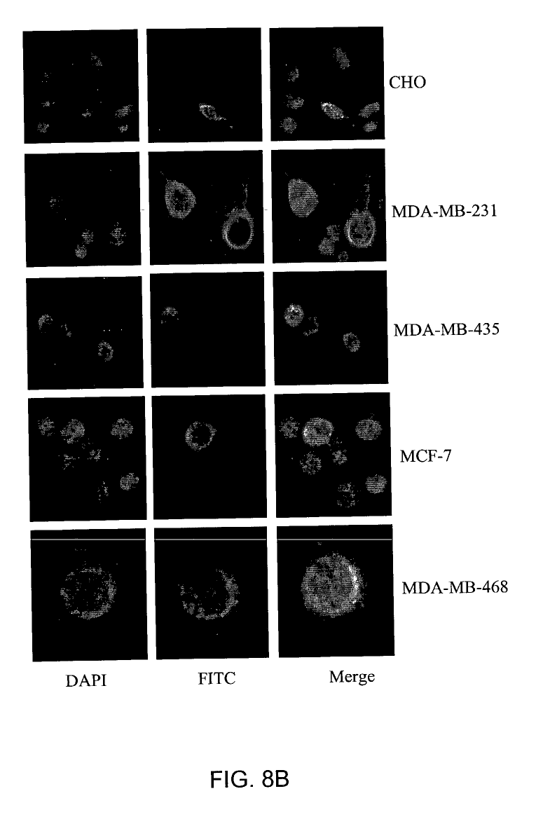

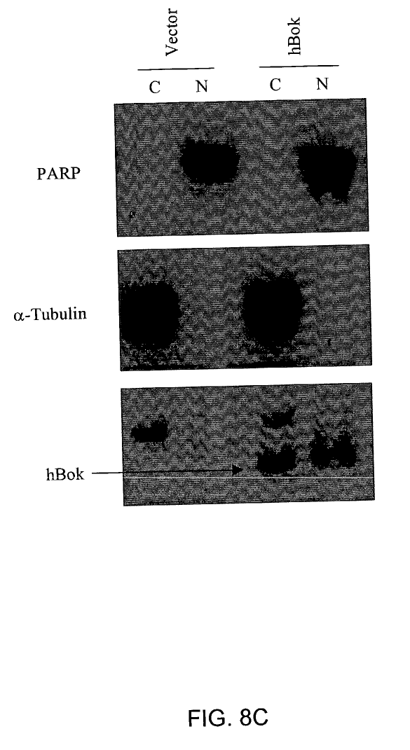

A putative NES ⁶⁹TVLLRLGDELEM⁷⁸ (SEQ ID NO: 36) is present at the N-terminus of hBok (FIG. 8A), suggesting that hBok may be localized in the nucleus. Therefore, it was investigated whether active transport events between the cytosol and nucleus are involved in regulating hBok activity. Indirect immunofluorescent staining of cultured CHO cells and the breast cancer cell lines MDA-MB-231, MDA-MB-435, MCF-7 and MDA-MB-468, each transiently transfected with Flag-tagged hBok and harvested 18-24 h after transfection, clearly detects hBok in the nucleus of all these cells (FIG. 8B). The concentration of nuclear hBok appeared to be higher in MDA-MB-468, and CHO cells than in MDA-MB-231, MDA-MB-435 and MCF-7 cells. In addition western blot analysis of both nuclear and cytoplasmic fractions from HEK293T cells harvested 24 h after transient transfection with the pcDNA3 control vector and the vector-expressing hBok showed hBok in the nucleus (FIG. 8C). A band was also detected only in the cytoplasmic fraction of both samples. This band is larger in size than that of hBok and is most likely the result of a non-specific interaction.

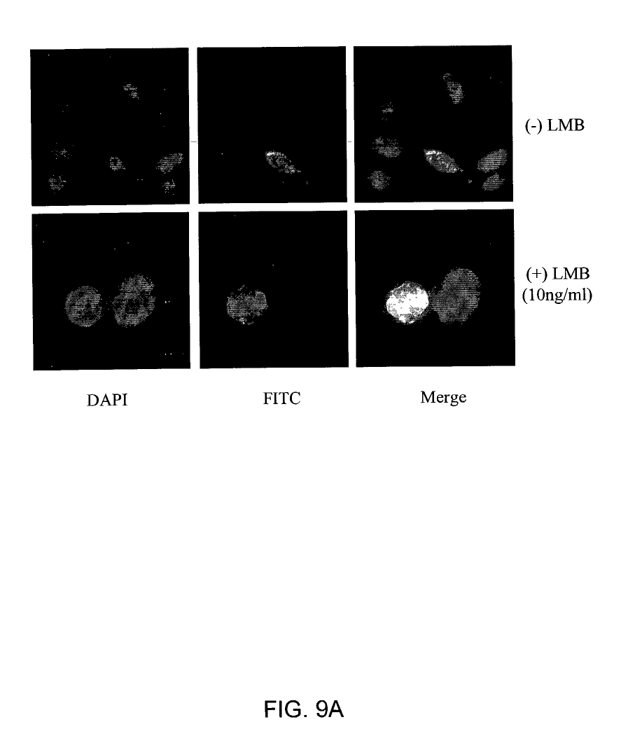

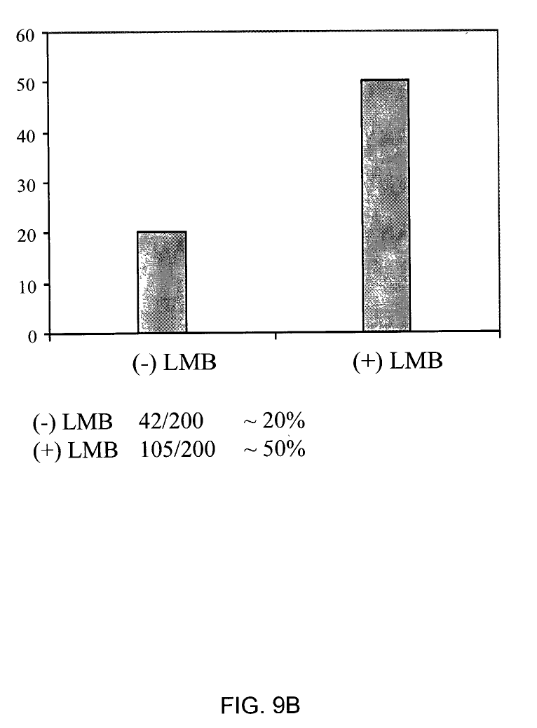

Thus, hBok shuttles between the nucleus and cytoplasm of the cells tested. If hBok is a nuclear protein, Leptomycin B (LMB) should sequester hBok in the nucleus. Leptomycin B is an unsaturated, branched-chain fatty acid identified as a specific inhibitor of the nuclear export of proteins that contain classical leucine-rich NES's, since it binds directly to the transporter protein CRM1 and blocks the binding of CRM1 to proteins containing the NES (Nishi et al., 1994). Exposing CHO cells transiently transfected with Flag-tagged hBok to LMB (10 ng/mL) for approximately 6 h resulted in an accumulation of the tagged hBok in the nucleus (FIG. 9A). Although Flag-tagged hBok was also detected in the nucleus of transfected cells not exposed to LMB, its nuclear concentration was significantly lower (FIG. 9B) This result clearly demonstrates that nuclear cytoplasm shuttling plays an important part in the hBok-induced apoptotic pathway.

### Example 7

### The NES Mutant of hBok Demonstrates Enhanced Apoptotic Activity

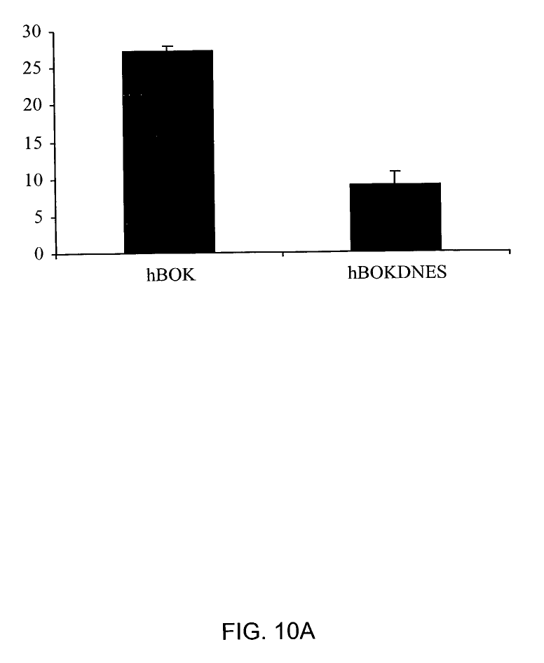

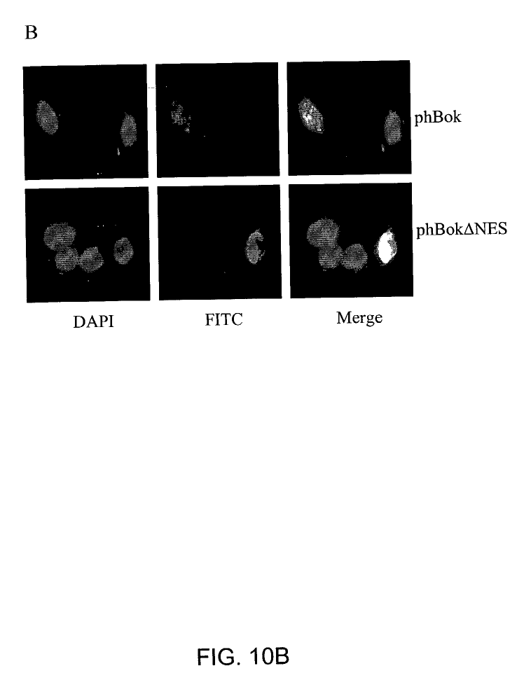

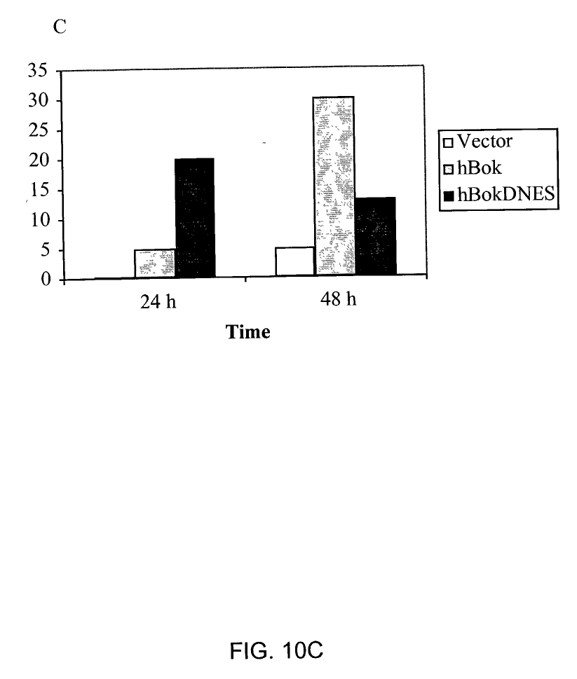

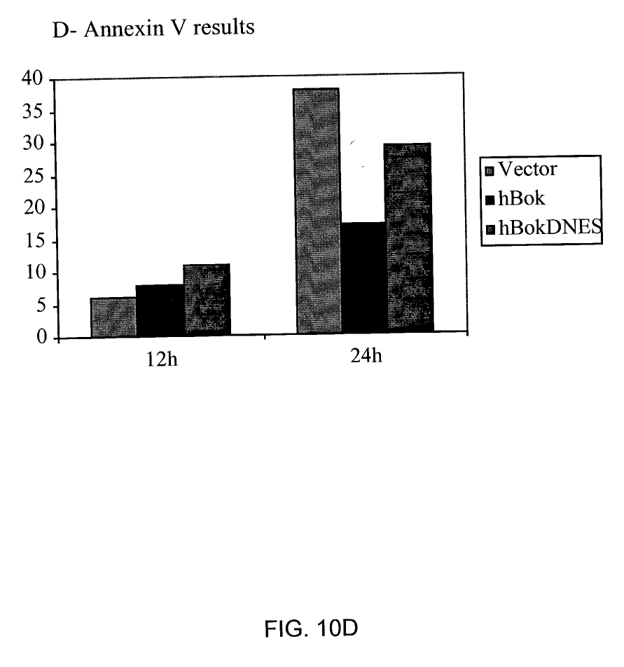

If nuclear localization of hBok is important for its proapoptotic activity, in specific non-limiting embodiments of the present invention the sequestering hBok in the nucleus might alter its proapoptotic activity. The 10 residue region of hBok that is responsible for its nuclear export comprises two leucine residues (71 Leu and 73 Leu) that are characteristic of a typical CRM1-dependent NES residues (Scott et al., 2000). A mutant was generated in which, presumably, the critical leucine residues in the putative NES sequence (71 Leu and 73 Leu) were substituted with alanine residues. (The amino acid residues ⁷¹LRL⁷³ were substituted with AAA). In a specific embodiment, the following polynucleotide sequence encoded the mutant amino acids: CTGGCGGCGGCGGGCGAT (SEQ ID NO: 35). HEK293T cells were transiently transfected with either the NES mutant or wild type hBok, and the ability of the mutant to kill HEK293T transfectants was compared to that of wild type using the luciferase viability assay. The NES hBok mutant demonstrated a greater ability to kill HEK293T cells when compared to wild type (FIG. 10A). The cell-killing effect of both the NES mutant and wild type hBok was specific because transfection of the vector had minimal effect on cell survival. Similar results were observed with the breast cancer cell lines MDA-MB 468, MDA-MB 435, MDA-MB 231 and MCF-7. Since mutating the putative NES of hBok enhances its proapoptotic activity, this raised the question whether this activity was a direct effect of its nuclear sequestration. To address this question, the nuclear concentration of wild type hBok and hBokΔNES mutant was compared in CHO transfectants by immunocytochemistry. The results show that disruption of the NES of hBok results in hBok being sequestered in the nucleus (FIG. 10B) To determine if sequestration of hBok in the nucleus of cells enabled hBok to induce apoptosis at a faster rate, the TUNEL assay was used to determined the onset of apoptosis in CHO cells transfected with either wild type hBok or the NES mutant (FIG. 10C). The results clearly demonstrate that the NES mutant hBok induces apoptosis at a faster rate than wild type. These results indicate that the NES mutant of hBok is not only a more potent inducer of apoptosis than wild type, but it also induces apoptosis at a faster rate.

One of the earliest morphological changes in apoptosis is the loss of plasma membrane. In apoptotic cells, the membrane-phospholipid phosphatidylserine (PS) is translocated from the inner to the outer leaflet of the plasma membrane, exposing PS to the external cellular environment. Annexin V is a 35-36 kDa Ca²⁺ dependent phospholipid-binding protein that has a high affinity for PS and binds to cells with exposed PS. Annexin V conjugated to the fluorochrome Phycoerythrin (PE) was used to serve as a sensitive probe for flow cytometric analysis of cells undergoing apoptosis.

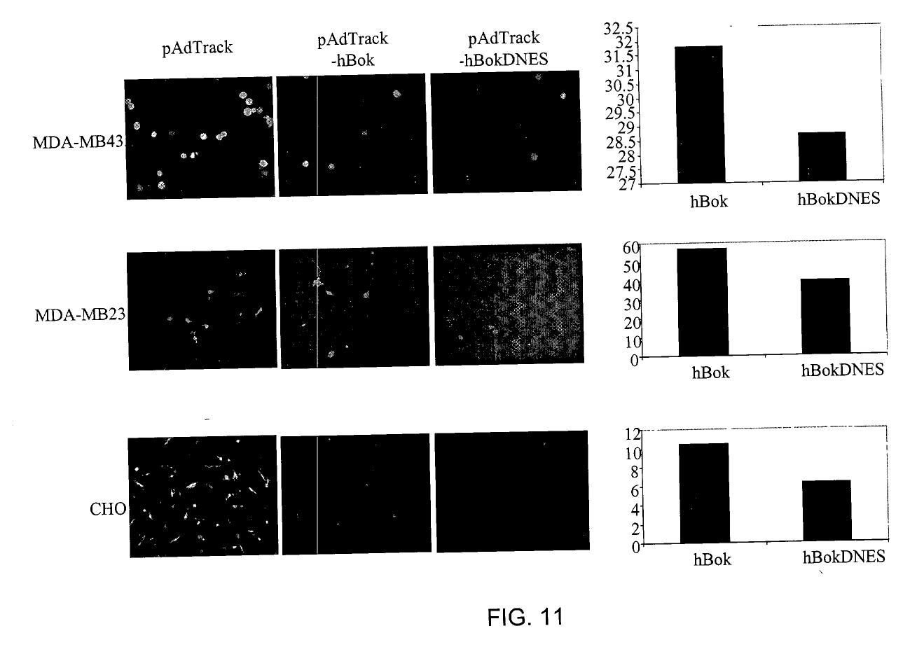

To further demonstrate the difference in apoptotic activity between wild type hBok and the hBokΔNES mutant, both cDNAs were cloned into the pADTrack vector in order to independently express the GFP and the hBok proteins from the same vector (FIG. 11). The breast cancer cell lines MDA-MB-231 and MDA-MB-435 were transfected together with the CHO cells. The results clearly show once again that the hBokΔNES mutant demonstrates a higher degree of potency in its ability to kill these cells.

### Example 8

### Regulation of BCL-2 Family Members by Phosphorylation and Bok Phosphorylation Mutants

The Bcl-2 family includes both pro-and anti-apoptotic proteins, and it has been proposed that these proteins regulate cell death by forming heterodimers (Reed, 1996). The relative concentrations of these two groups of proteins determine whether the cell survives or undergoes apoptosis (Gross et al., 1999; Oitvai and Korsmeyer, 1994; Allen et al., 1998). Recently, it has been documented that phosphorylation of the anti-and pro-apoptotic proteins influences their activity. The phosphorylation of Bcl-2 at ser70 by activated c-Jun N-terminal kinase (JNK) (Deng et al,. 2001) or protein kinase (Ito et al,. 1997; Ruvolo et al, 1998) induces its antiapoptotic activity. On the other hand, phosphorylation of Bcl-2 by anti-cancer drugs such as taxol induces apoptosis by inactivating Bcl-2 (Cheng et al., 2001; Basu et al., 2000; Basu et al., 2000; Yamamoto et al., 1999; Haldar et al., 1998). Phosphorylation of the BH3 domain only proapoptotic Bcl-XL/Bcl-2-Associated Death Promoter (Bad) by either the mitogen activated protein (MAPK) kinase and/or the phosphatidylinositide-3-OH-kinase (P13K) dependent signaling pathways blocks its ability to heterodimerize with the antiapoptotic proteins Bcl-XL and Bcl-2 resulting in diminished cell killing (Hsu et al., 1997; Zha et al., 1996; Scheid et al., 1999; Pastorino et al., 1999). Hence, the net apoptotic signal delivered by the Bcl-2 family members may depend not only on the relative ratios of pro- and anti-apoptotic members but also on the degree of phosphorylation.

The N-terminal end of hBok comprises multiple kinase recognition motifs, which raises the possibility that phosphorylation may play an important role in the regulation of hBok function. Since it is known that phosphorylation influences the function of both anti- and proapoptotic members of the BCl-2 family, such as Bcl-2, Bad and Bik (Ruvolo et al., 2001; Dramsi et al., 2002; Verma et al., 2001) the MAP kinase and protein kinase C recognition motifs are characterized. It is determined if loss of these motifs influences the proapoptotic activity of hBok. The 21S amino acid residue of the putative MAP kinase motif and the 23T amino acid residue of the putative protein kinase C substrate motifs were substituted with either alanine or glutamic acid, and the apoptotic activity of these mutants was tested in breast tumor cells growing in tissue culture. The alanine mutants did demonstrate a greater killing ability than wild type.

### Example 9

### Clinical Trials

The choice of Bok in the present invention is of clinical significance since Bcl-2, an inhibitor of apoptosis, is overexpressed in many different human tumors (Liang et al., 1995; Tsujimoto and Croce, 1986; Hollstein et al., 1991; McDonnell et al., 1992; Haldar et al., 1994; Ikegaki et al., 1994; Sinicrope et al., 1995), and the activity of hBok is not antagonized by Bcl-2. The uniqueness of Bok was further expanded when the present inventors detected it in the nucleus of cancer cells, suggesting that, unlike with other members of the BCL-2 family, nuclear localization of Bok is important for its function. That is, nuclear sequestration of hbok resulted in both a faster onset of apoptosis and a greater apoptotic activity. The present Examples provide the first data to identify hBok in the nucleus and establish a functional link between nuclear localization of hBok and its apoptotic function.

In specific embodiments, this nuclear localization capability, or its lack thereof, is exploited for purposes of cancer therapy. As shown in the Examples, it was clearly demonstrated that a Bok nucleic acid sequence had the ability to inhibit ovarian cancer in an appropriate animal model and to enhance survival of the tumor bearing mice. Furthermore, Bok is effective against a variety of cancer cell lines (such as breast, prostate, and pancreatic). This antitumor activity demonstrates that Bok is useful in methods and compositions for the treatment of tumors. A skilled artisan recognizes based on the teachings herein that multiple specific embodiments of Bok sequences are useful for such treatment, including rBok, hBok, and any mutant thereof which comprise activity against tumor cells in vitro and/or in vivo. In particular embodiments, the Bok mutant comprises a defective nuclear export sequence. A specific example of a useful Bok sequence is SEQ ID NO: 34.

In one embodiment of the present invention, toxicity studies are performed in immuno-competent mice using a Bok nucleic acid sequence coupled with either a viral or non-viral delivery system. Demonstration that the effective dose does not have significant toxicity in the immuno-competent mice indicates the Bok gene therapy can be moved into clinical trials. In addition to the ovarian model used in this invention, the antitumor activity of Bok is tested in other cancer models, such as breast cancer and prostate cancer.

This example is concerned with the development of human treatment protocols using a Bok mutant protein, peptide, or polypeptide or a nucleic acid encoding Bok protein, peptide, or polypeptides, alone or in combination with other anti-cancer drugs. The a Bok protein, peptide, or polypeptide or a nucleic acid encoding the Bok protein, peptide, or polypeptides, and anti-cancer drug treatment will be of use in the clinical treatment of various cancers involving, for example, ovarian, breast, and prostate. As described in Example 1, the Bok treatment is for cancer irrespective of its p53 and/or caspase 3 phenotype. Such treatment will be particularly useful tools in anti-tumor therapy, for example, in treating patients with ovarian, breast, prostate, and other cancers that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the Bok protein, peptide, or polypeptide or a nucleic acid encoding a Bok protein, peptide, or polypeptides, in clinical trials.

Patients with ovarian, breast, prostate, or other cancers chosen for clinical study will typically be at high risk for developing the cancer, will have been treated previously for the cancer which is presently in remission, or will have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to a Bok protein, peptide, or polypeptide or a nucleic acid encoding a Bok protein, peptide, or polypeptides, and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), and in the cells (Bok proteins, peptides or polypeptides or nucleic acids encoding the same) may be assessed and recorded.

In the same procedure, the Bok protein, peptide, or polypeptide or a nucleic acid encoding the Bok protein, peptide, or polypeptide, may be administered alone or in combination with another anti-cancer drug. The administration may be, for example, in the pleural/peritoneal cavity, directly into the tumor, or in a systemic manner. The starting dose may be, for example, 0.5 mg/kg body weight. Three patients may be treated at each dose level in the absence of grade>3 toxicity. Dose escalation may be done by, for example, 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the Bok protein, peptide, or polypeptide or a nucleic acid encoding the Bok protein, peptide, or polypeptide, and the lot of anti-cancer drug exceed 5 EU/kg for any given patient.

The Bok protein, peptide, or polypeptide or a nucleic acid encoding the Bok protein, peptide, or polypeptides, and/or the other anti-cancer drug combination, may be administered over a short infusion time or at a steady rate of infusion over a 7 to 21 day period. The Bok protein, peptide, or polypeptide or a nucleic acid encoding the Bok protein, peptide, or polypeptide, infusion may be administered alone or in combination with the anti-cancer drug. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the Bok protein, peptide, or polypeptide or a nucleic acid encoding the Bok protein, peptide, or polypeptide, in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phopshorylated forms) and Akt (phosphorylated and non-phosphorylated forms), p185, etc.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myclosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, ki67 and Tunel assay to measure apoptosis, Akt) and in the cells (Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. A urinalysis may be performed every 4 weeks.

Clinical responses may be defined by acceptable measure. For example, a complete response may be defined by the disappearance of all measurable disease for at least a month. A partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### Example 10

### Significance of the Present Invention

This study is the first to identify hBok in the nucleus and establish a functional link between nuclear localization of hBok and its apoptotic function. This observation differed from earlier results that showed that deletion mutations within the BH3 domain of rBok failed to enhance the apoptotic activity of the protein (Hsu and Hsueh, 1998). A possible explanation for this difference is that two of the rBok mutants did not disrupt the NES. Although the NES was disrupted in the third mutant by being replaced with glutamic acid residues, this mutant too, failed to demonstrate an enhanced apoptotic activity, raising the possibility that replacing the critical residues of the NES with alanine rather than glutamic acid might result in enhanced apoptotic activity. There is no published data to date that links nuclear localization of pro-apoptotic BCL-2 family member to its apoptotic function. The observation that nuclear localization is important for hBok apoptotic activity is novel, and it is the first time that a biological function has been associated with the nuclear localization of a member of the Bax family of proapoptotic Bcl-2 proteins.

Thus, the present invention is useful for treating a wide variety of cancers irrespective of their Bcl-2 levels. In a specific embodiment, a mouse tumor model is utilized to test a tumor-specific promoter to drive the expression of hBok and the mutant.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

PATENTS

| | Issue Date |
|---|---|
| U.S. Pat. No. 4,162,282 | Jul. 24, 1979 |
| U.S. Pat. No. 4,310,505 | Jan. 12, 1982 |
| U.S. Pat. No. 4,533,254 | Aug. 06, 1985 |
| U.S. Pat. No. 4,554,101 | Nov. 19, 1985 |
| U.S. Pat. No. 4,728,575 | Mar. 01, 1988 |
| U.S. Pat. No. 4,728,578 | Mar. 01, 1988 |
| U.S. Pat. No. 4,737,323 | Apr. 12, 1988 |
| U.S. Pat. No. 4,921,706 | May 01, 1990 |
| U.S. Pat. No. 5,399,346 | Mar. 21, 1995 |
| U.S. Pat. No. 5,401,511 | Mar. 28, 1995 |
| U.s. Pat. No. 5,432,260 | Jul. 11, 1995 |
| U.S. Pat. No. 5,603,872 | Feb. 18, 1997 |
| U.S. Pat. No. 5,786,214 | Jul. 28, 1998 |
| U.S. Pat. No. 5,849,718 | Dec. 15, 1998 |
| U.S. Pat. No. 5,871,727 | Feb. 16, 1999 |
| U.S. Pat. No. 5,879,703 | Mar. 09, 1999 |
| U.S. Pat. No. 5,889,155 | Mar. 30, 1999 |
| U.S. Pat. No. 5,939,277 | Aug. 17, 1999 |
| U.S. Pat. No. 6,043,055 | Mar. 28, 2000 |
| U.S. Pat. No. 6,107,090 | Aug. 22, 2000 |
| U.S. Pat. No. 6,222,017 | Apr. 24, 2001 |
| EPO 0273085 | Jul. 06, 1988 |
| GB 2193095 | Feb. 03, 1988 |
| PCT/US85/01161 | |
| PCT/US89/05040 | |
| WO 98/0748 | |
| WO 99/18933 | |

### PUBLICATIONS

Allen, R. T., M. W. Cluck, and D. K. Agrawal, Mechanisms controlling cellular suicide: role of Bcl-2 and caspases. Cellular & Molecular Life Sciences, 1998. 54(5): p. 427-45.

Bangham, et al., “Diffusion of univalent Ions across the Lamellae of Swollen Phospholipids” J. Mol. Biol., 13:238-252, 1965.

Basu, A., S. A. You, and S. Haldar, Regulation of Bcl2 phosphorylation by stress response kinase pathway. International Journal of Oncology, 2000. 16 (3): p. 497-500.

Cheng, S. C., D. Luo, and Y. Xie, Taxol induced bcl-2 protein phosphorylation in human hepatocellular carcinoma qgy-7703 cell line. Cell Biology International, 2001. 25: p. 261-265.

Deamer and P. Uster, “Liposome Preparation: Methods and Mechanisms,” in Liposomes (M. Ostro, ed.), Marcel Dekker, Inc., New York (1983), pp. 27-52.

Deng X, Xiao L, Lang W, Gao F, Ruvolo P, May W S Jr. Novel role for JNK as a stress-activated Bcl2 kinase. J Biol Chem. June 2001 29; 276(26):23681-8.

Dramsi, S., M. P. Scheid, A. Maiti, P. Hojabrpour, X. Chen, K. Schubert, and D. R. Goodleft. 2002 Identification of a novel phosphorylation site, Ser-170, as a regulator of bad pro-apoptotic activity. J. Biol. Chem. 277:6399-6405.

Fraley and Fornari Kaplan, “Entrapment of a bacterial plasmid in phospholipid vesicles:potential for gene transfer,” Proc. Nat'l. Acad. Sci. USA 76:3348-3352, 1979.

Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.

Gregoriadis G. and Davis C. “Stability of liposomes in vivo and in vitro is promoted by their cholesterol content and the presence of blood cells,” Biochem Biophys Res Commun., 89(4):1287-1293, 1979.

Gregoriadis, DRUG CARRIERS IN BIOLOGY AND MEDICINE, G. Gregoriadis (ed.), 1979, pp. 287-341.

Gross, A., J. M. McDonnell, and S. J. Korsmeyer, BCL-2 family members and the mitochondria in apoptosis. Genes & Development, 1999. 13(15): p. 1899-911.

Haldar, S., A. Basu, and C. M. Croce, Serine-70 is one of the critical sites for drug-induced Bcl2 phosphorylation in cancer cells. Cancer Research, 1998. 58(8): p. 1609-15.

Hsu S Y. Hsueh A J. A splicing variant of the Bcl-2 member Bok with a truncated BH3 domain induces apoptosis but does not dimerize with antiapoptotic Bcl-2 proteins in vitro. Journal of Biological Chemistry. 273(46):30139-46, 1998.

Hsu S Y. Kaipia A. McGee E. Lomeli M. Hsueh A J. Bok is a pro-apoptotic Bcl-2 protein with restricted expression in reproductive tissues and heterodimerizes with selective anti-apoptotic Bcl-2 family members. Proceedings of the National Academy of Sciences of the United States of America. 94(23): 12401-6, 1997.

Hsu, S Y, Hsueh, A J W. Tissue-Specific Bcl-2 Protein Partners in Apotosis: An Ovarian Paradigm. Physiology Reviews, 80(2): 593-614, 2000.

Hsu, S Y, Hsueh, A J W. Intracellular mechanisms of ovarian cell apoptosis. Molecular and Cellular Endocrinology, 145: 21-25, 1998.

Inohara, N., D. Ekhterae, J. Garcia, R. Carrio, G. Merino, A. Merry, S. Chen and G. Nunez 1998 Mtd, a novel Bcl-2 family member activates apoptosis in the absence of heterodimerization with Bcl-2 and Bcl-XL. J. Biol. Chem. 273:8705-8710.

Ito, T., et al., Bcl-2 phosphorylation required for anti-apoptosis function. Journal of Biological Chemistry, 1997. 272(18): p. 11671-3.

Kaneda et al., “Increased expression of DNA cointroduced with nuclear protein in adult rat liver,” Science, 243:375-378, 1989.

Kaneda et al., “Introduction and expression of the human insulin gene in adult rat liver,” J Biol Chem., 264(21):12126-12129, 1989.

Kato et al., “Expression of hepatitis B virus surface antigen in adult rat liver. Co-introduction of DNA and nuclear protein by a simplified liposome method,” J Biol Chem., 266(6):3361-3364, 1991.

Leo C P. Hsu S Y. Chun S Y. Bae H W. Hsueh A J. Characterization of the antiapoptotic Bcl-2 family member myeloid cell leukemia-1 (Mcl-1) and the stimulation of its message by gonadotropins in the rat ovary Endocrinology. 140(12):5469-77, 1999.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al., “Liposomes as carriers for in vivo gene transfer and expression,” Methods Enzymol., 149:157-176, 1987.

Oitvai, Z. N. and S. J. Korsmeyer, Checkpoints of Dueling Dimers foil Death Wishes. Cell, 1994. 79: p. 189-192.

Perales et al., “Gene transfer in vivo: sustained expression and regulation of genes introduced into the liver by receptor-targeted uptake,” Proc. Natl. Acad. Sci. USA, 91:4086-4090, 1994.

Reed, J. C., Mechanisms of Bcl-2 family protein function and dysfunction in health and disease. Behring Institute Mitteilungen, 1996(97): p. 72-100.

Ruvolo, P. P., X. Deng, and W. S. May. 2001 Phosphorylation of Bcl2 and regulation of apoptosis. Leukemia 15:515-522.

Ruvolo, P. P., et al., A functional role for mitochondrial protein kinase Calpha in Bcl2 phosphorylation and suppression of apoptosis. Journal of Biological Chemistry, 1998. 273(39): p. 25436-42.

Pastorino, J. G., M. Tafani, and J. L. Farber, Tumor necrosis factor induces phosphorylation and translocation of BAD through a phosphatidylinositide-3-OH kinase-dependent pathway. Journal of Biological Chemistry, 1999. 274(27): p. 19411-6.

Scheid, M. P., K. M. Schubert, and V. Duronio, Regulation of bad phosphorylation and association with Bcl-x(L) by the MAPK/Erk kinase. Journal of Biological Chemistry, 1999. 274(43): p. 31108-13.

Szoka and Papahadjopoulos, “Procedure for Preparation of Liposomes With Large Internal Aqueous Space . . . ”, Proc. Natl. Acad. Sci., 75:4194-4198, 1978.

Verma, S., L. J. Zhao, and G. Chinnadurai. 2001 Phosphorylation of the pro-apoptotic protein BIK: mapping of phosphorylation sites and effect on apoptosis. J. Biol. Chem. 276:4671-4676.

Wagner et al., Science, 260:1510-1513, 1990.

Wong et al., “Appearance of beta-lactamase activity in animal cells upon liposome-mediated gene transfer,” Gene., 10(2):87-94, 1980.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Yamamoto, K., H. Ichijo, and S. J. Korsmeyer, BCL-2 is phosphorylated and inactivated by an ASK1/Jun N-terminal protein kinase pathway normally activated at G(2)/M. Molecular & Cellular Biology, 1999. 19(12): p. 8469-78.

Zha, J., et al., Serine phosphorylation of death agonist BAD in response to survival factor results in binding to 14-3-3 not BCL-X(L). [see comments] Cell, 1996. 87(4): p. 619-28.

Zhang, H, Holzgreve, W, De Geyter, C. Evolutionary conserved Bok proteins in Bcl-2 family. FEBS Letters, 480: 311-313, 2000.

All of the compositions and/or methods disclosed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method of inhibiting cell proliferation comprising contacting a cell with a Bok polypeptide in an amount effective to inhibit the cell proliferation.

2. The method of claim 1, wherein said Bok polypeptide is introduced into said cell by the direct introduction of said Bok polypeptide.

3. The method of claim 1, wherein the Bok polypeptide is a modified Bok polypeptide.

4. The method of claim 1, wherein the Bok polypeptide is introduced into the cell through the introduction of a Bok-encoding polynucleotide.

5. The method of claim 4, wherein the Bok-encoding polynucleotide encodes a modified Bok polypeptide.

6. The method of claim 4, wherein the polynucleotide is a deoxyribonucleic acid molecule.

7. The method of claim 4, wherein said Bok-encoding polynucleotide further comprises at least one regulatory sequence.

8. The method of claim 7, wherein said regulatory sequence is a promoter.

9. The method of claim 4, wherein said Bok-encoding polynucleotide is comprised in a vector.

10. The method of claim 9, wherein said vector is a plasmid.

11. The method of claim 9, wherein said vector is a viral vector.

12. The method of claim 9, wherein said vector is a non-viral vector.

13. The method of claim 4, wherein said Bok-encoding polynucleotide is comprised with a nonviral gene delivery system.

14. The method of claim 13, wherein said polynucleotide is complexed with a lipid.

15. The method of claim 13, wherein said polynucleotide is comprised in a liposome.

16. The method of claim 1, wherein said cell is a tumor cell.

17. The method of claim 16, wherein said tumor cell is in a tumor.

18. The method of claim 17, further comprising treating an individual having the tumor with surgery.

19. The method of claim 17, wherein said tumor is in an animal.

20. The method of claim 19, wherein said animal is a human.

21. The method of claim 1, wherein said Bok polypeptide has tumor suppressor activity.

22. The method of claim 1, further comprising treating the cell with a second agent.

23. The method of claim 1, further defined as comprising contacting the Bok polypeptide with the cell by injecting a Bok polynucleotide encoding the polypeptide into an animal comprising the cell.

24. The method of claim 23, wherein said injection is intraperitoneally.

25. A method of treating a proliferative cell disorder in an individual comprising the step of administering to said individual a Bok composition in an amount effective to treat said disorder.

26. The method of claim 25, wherein said Bok composition is a polypeptide, wherein the polypeptide is introduced into the cell by the direct introduction of the Bok polypeptide.

27. The method of claim 26, wherein the Bok polypeptide is a modified Bok polypeptide.

28. The method of claim 25, wherein the Bok composition is a Bok polypeptide, wherein the Bok polypeptide is introduced into the cell through the introduction of a Bok-encoding polynucleotide.

29. The method of claim 28, wherein the Bok-encoding polynucleotide encodes a Bok modified polypeptide.

30. The method of claim 28, wherein the polynucleotide is a deoxyribonucleic acid molecule.

31. The method of claim 28, wherein said Bok-encoding polynucleotide further comprises at least one regulatory sequence.

32. The method of claim 31, wherein said regulatory sequence is a promoter.

33. The method of claim 28, wherein said Bok-encoding polynucleotide is comprised in a vector.

34. The method of claim 33, wherein said vector is a plasmid.

35. The method of claim 33, wherein said vector is a viral vector.

36. The method of claim 33, wherein said vector is a nonviral vector.

37. The method of claim 28, wherein said Bok-encoding polynucleotide is comprised with a lipid.

38. The method of claim 28, wherein said polynucleotide is complexed with said lipid.

39. The method of claim 28, wherein said polynucleotide is comprised in a liposome.

40. The method of claim 25, wherein said proliferative cell disorder is cancer.

41. The method of claim 25, further comprising treating the cell with a second agent.

42. The method of claim 40, further comprising treating the cancer with surgery.

43. The method of claim 26, wherein said Bok polypeptide has tumor suppressor activity.

44. The method of claim 25, further defined as comprising contacting a cell with a polynucleotide encoding a Bok polypeptide by injecting the polynucleotide into the individual comprising the cell.

45. The method of claim 44, wherein said injection is intraperitoneally.

46. A kit for treatment of an individual with cancer, wherein the kit is housed in a suitable container, comprising:
a Bok composition; and
a pharmaceutical carrier for said composition.

47. The kit of claim 46, wherein said Bok composition is a Bok polypeptide.

48. The kit of claim 47, wherein the Bok polypeptide is a modified Bok polypeptide.

49. The kit of claim 46, wherein said Bok composition is a Bok polynucleotide.

50. The kit of claim 49, wherein said Bok polynucleotide encodes a modified polynucleotide.

51. A method of treating cancer in an individual having the cancer, comprising contacting a cancer cell of the individual with a therapeutically effective amount of a polynucleotide encoding a Bok polypeptide, wherein the polynucleotide is comprised in a liposome.
